(19) United States
(12) Patent Application Publication
(10) Pub. No.: US 2008/0318269 A1
(43) Pub. Date: Dec. 25, 2008

(54) Devices and Methods for the Analysis of Biofilm

(76) Inventors: Merle E Olson, Calgary (CA); Howard Ceri, Calgary (CA)

Correspondence Address: WILLIAM J BUNDREN;THE LAW OFFICE OF WILLIAM J BUNDREN, 734 LaRue Road, Millersville, MD, 21108, US

(21) Appl. No.: 11/996,480

(22) PCT Filed: Jul. 22, 2006
(86) PCT No.: PCT/CA2006/001226

§ 371 Date: Aug. 19, 2008

Related U.S. Application Data
(60) Provisional application No. 60/701,858, filed on Jul. 22, 2005.

(51) Int. Cl. C12Q 1/06; C12Q 1/02

(52) U.S. Cl. 435/39; 435/29

(57) ABSTRACT

This invention is a diagnostic plate that can be used to select antibiotic combinations with efficacy against microorganisms growing as a biofilm. The plate allows growth of biofilm on a plurality of projections, and the subsequent simultaneous challenge of biofilms on all projections of the plate to independent concentrations and combinations of anti-biofilm agents. Resistance of microorganisms to antibiotics is higher when they grow as a biofilm, as compared to when they grow in a planktonic state which is usually used to determine their level of antibiotic sensitivity. Growth of microorganims that slough off the biofilm in the anti-biofilm agent challenge determines the Minimum Inhibitory Concentration (MIC) which relates to sensitivity of the microorganisms in a planktonic state. Growth of any surviving microorganims from the biofilm in a subsequent recovery step determines the Minimal Biofilm Eradication Concentration (MBEC) which relates to the sensitivity of the microorganisms growing as a biofilm. Enumeration of the surviving microorganims in the recovery step determines the Minimum Biocidal Concentration (MBC).

This application is claims priority from U.S. Provisional Application No. 60/701,858 filed on Jul. 22, 2005.

### I. FIELD OF THE INVENTION

This invention relates to methods and apparatus for the analysis of biofilms, and to determining microbial sensitivity to anti-microbial reagents.

This invention relates to methods and devices apparatus for the analysis of biofilms, and to determining microbial sensitivity to anti-microbial or anti-biofilm reagents, preferably combinations of anti-biofilm reagents, such as antibiotics or biocides.

### II. BACKGROUND OF THE INVENTION

The characterization of microorganisms has traditionally employed methods of batch culture studies, where the organisms exist in a dispersed, or planktonic state (1). Over the past 25 years, it has been recognized that the major component of the bacterial biomass in many environments are sessile bacteria (2). These studies have indicated that most microorganisms are capable of growth in biofilms, and that the growth of organisms in biofilms is physically and physiologically different than growth of the same organism in batch culture. These differences contribute to observed alterations in both the pathogenesis of these organisms and their susceptibility to antimicrobial agents (1,3).

Selecting antibiotics and combinations of antibiotics for treating biofilm infections continues to rely on minimal inhibitory concentration (MIC) assays despite the recognized lack of efficacy of these tests. Some have suggested the use of biofilm inhibitory concentrations (BIC) (Moskowitz, et al.; J. Clin. Microbiology, 42:1915-1922 (May 2004)), but the evidence suggests that both BIC and MIC address planktonic bacteria, not sessile bacteria. For example, Moskowitz et al. use centrifugation in their process, and do not remove the vast majority of cells derived from the challenged biofilm, therefore resulting in an assay for the planktonic bacteria alone, or an assay of only a portion of the biofilm. The assay therefore may miss viable cells left on the pegs, therefore leading to a potentially inaccurate conclusion. Further, the prior art typically grows the biofilm in a static (non-flowing) environment, which sometimes affects the results.

In contrast, the present invention uses sonication or re-growing biofilm on a separate recovery plate in its processing so that the complete, intact biofilm can be obtained and assayed. Also, the processes of the present invention include growing the biofilm under dynamic or flowing conditions, and neutralizing the anti-microbials, both of which individually and collectively fortify any assay results.

Therefore a need exists for improved processing and assaying devices and methods for selecting effective compositions against biofilm.

### III. SUMMARY OF THE INVENTION

The invention comprises improved methods and devices for the selection of one or more active agents, either alone or in combination, effective against biofilm. The biofilm may be any biofilm, e.g., those formed from bacteria, fungi, or algae, viruses, and parasites; or a microorganism that is incorporated within a biofilm as it is formed. The devices and methods of the present invention are also useful in assaying and treating mixed biofilms, e.g., containing more than one bacterial, viral, fungal, parasitic, or algal biofilm.

The present invention provides a panel of individual and/or combined active agents for selecting a composition containing one or more active agents with efficacy against a biofilm.

The invention also provides an in vitro assay tailored to the presence of a biofilm, namely an assay based on determining the minimum biofilm eliminating concentration (MBEC). In preferred embodiments of the invention, the devices and methods provide any combination of MBEC, minimum inhibitory concentration (MIC), and minimum biocidal concentration (MBC) values.

The devices and methods of the present invention are improved over prior art devices in one or more of the following: the device and process involve testing intact biofilm; using sonication to remove the intact biofilm; the devices and process apply to a wider range of biofilms, e.g., fungal, etc.; the anti-biofilm agent covers a wider range of agents, including biocides, etc.; the devices and methods are high-throughput and therefore more efficient and cost effective; and growing the biofilm is improved, involving increased understanding and application of process conditions to enhance biofilm growth.

Microbial biofilms exist in a number of medical, veterinary, agricultural and industrial systems, processes, processing equipment, and surfaces. The organisms present on these surfaces include a number of pathogenic and nonpathogenic bacteria and fungi.

The methods and devices of the present invention may be used to degrade biofilms wherever they occur, e.g., in industrial processes where fouling occurs, e.g., de-fouling pulp and paper mill equipment, treating of a gas/oil pipe line, and decontaminating food processing equipment, or implanted medical devices, including catheters, hip implants, and cannulae. It is within the scope of this invention that the principles outlined here also apply to all biofilms in all circumstances in which they occur.

These and other aspects of the invention will be made apparent in the figures, description, and claims that follow.

### III. BRIEF DESCRIPTION OF THE DRAWINGS

FIG. 1 is a bottom view of plural biofilm adherent sites on a lid of a vessel.

FIG. 2 is a top view of a vessel for receiving the plural biofilm adherent sites of FIG. 1.

FIG. 3 is a side view, partly broken away, of the lid and vessel of FIGS. 1 and 2.

FIG. 4 is a flow diagram of the process steps is an exemplary embodiment of the invention.

FIG. 5 shows an example of a biofilm growth and formation process of the present invention.

FIG. 6 shows an example of a biofilm susceptibility assay of the present invention.

FIG. 7 shows an example of a process for recovering intact biofilm in accordance with the present invention.

FIG. 8 shows an example of a process for establishing MBEC and MIC determinations in accordance with the present invention.

FIG. 9 is a chart of the results of the experiment described in Example 6.

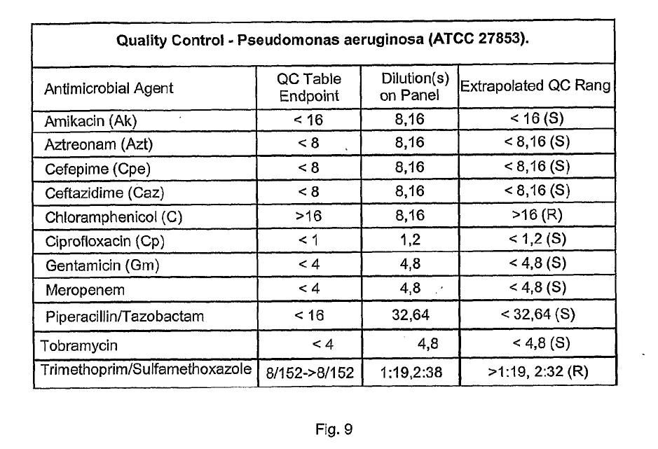

FIG. 10 shows the configuration of a challenge plate used in Example 7.

FIG. 11 shows the configuration of a challenge plate used in Example 10.

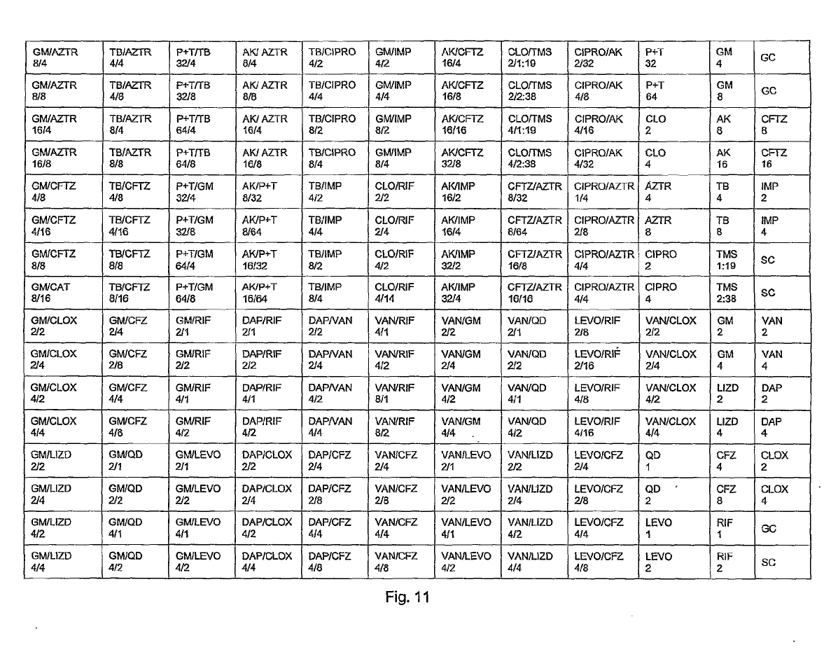

FIG. 12 is a chart of the MIC, MFC, and MBEC values determined biofilms.

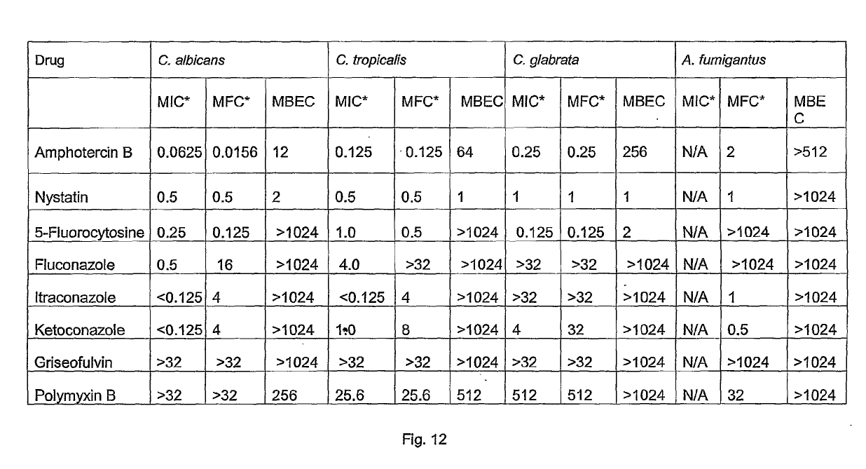

### IV. DETAILED DESCRIPTION OF THE INVENTION

The invention comprises improved methods and devices for selecting appropriate combinations of anti-biofilm agents for the treatment of biofilm. In preferred embodiments of the invention, the methods and devices provide diagnostic susceptibility testing and in the most preferred embodiments, provide MBEC, MBC, and MIC values in a single experiment. An embodiment of the invention may include a method and device for selecting synergistic combinations of active agents against specific biofilms or groups of biofilms.

The methods and devices of the present invention may also be used to select the appropriate biocide, combination of biocides, or composition that includes one or more biocides to treat biofilm wherever it may be found. In this aspect of the invention, compositions that includes one or more biocides refers to a composition that also includes one or more other active ingredients, including but not limited to surfactants and corrosion inhibitors.

An embodiment of the invention includes a device comprising plates pre-loaded with one or more pre-selected anti-biofilm agents against a specific biofilm or biofilms.

An embodiment of the invention includes methods for determining efficacy of anti-biofilm activity based on a pre-determined end point determination as desired by one skilled in the art. Exemplary end point determinations include, but are not limited to the following assays to quantify bacterial viability, change in optical density, direct bacterial counts, dye binding assays, metabolic activity assays, ATP assays, and live-dead staining assays.

In some embodiments of the invention, the method may also include one or more of the following: growing multiple or plural biofilms under conditions that promote the production of substantially uniform biofilms; screening the biological sample against a large group of active agents; selecting a subgroup of active agents; loading an assay device with multiple or plural active agents in the subgroup; growing biofilm from a specific patient's or subject's sample; screening the biofilm from the specific patient or subject against the subgroup of active agents; reading the results; determining the appropriate active agent or combination of active agents suitable for the particular biofilm; conducting a turbidity assay if the microorganism produces visible turbidity when growing (e.g. Pseudomonas); and conducting a plating assay if the microorganism does not grow with visible turbidity.

An embodiment of the invention includes rehydrating a species specific plate of preloaded antibiotics as the challenge plate to identify antibiotics with efficacy against the specific pathogen. Plates may be fiozen (no rehydration required), or lyophilized, freeze dried or vacuum dried.

An embodiment of the invention includes a well plate containing lyophilized antibiotic combinations that can be re-hydrated to be used in antibiotic susceptibility assay.

In a further aspect of the invention, the biofilm adherent sites are projections from a lid and incubating the biofilm includes suspending the projections in liquid growth medium in the channels while rocking or shaking the vessel so as to provide shear forces on the biofilm during growth of the biofilm. Preferably, the flow direction of the liquid growth medium is repeatedly reversed. In this aspect of the invention, the liquid growth medium may flow in channels of a vessel, and the direction of flow of the liquid growth medium is reversed by rocking of the vessel.

There is further provided, in accordance with an aspect of the invention, an apparatus for growing one or more biofilms, the apparatus comprising: a vessel including at least one channel for flow of liquid growth medium; plural or multiple biofilm adherent sites arranged in at least one row and having a support for supporting the biofilm adherent sites within the channel; and means to flow liquid growth medium along each channel in different directions across the plural biofilm adherent sites.

There is further provided, in accordance with an aspect of the invention, an apparatus for assaying one or more biofilms, the apparatus comprising: a vessel including at least one well, said vessel being configured to receive a lid comprising biofilm adherent sites; said vessel and lid being adapted and configured to provide MBEC, MIC, and MBC values for said biofilm.

An embodiment of the invention includes determining anti-microbial efficacy by establishing one or more endpoint determinations, said endpoint determinations, including but not limited to one or more of the following processes: change in optical density, direct bacterial counts, dye binding assays, metabolic activity assays, ATP assays, and live-dead staining assays.

In a further aspect of the invention, microorganisms are incubated to form a biofilm on plural or multiple biofilm adherent sites arranged in plural rows, with plural biofilm adherent sites in each row, while providing a flow of liquid growth medium across the plural biofilm adherent sites, and an assay made of the resulting biofilm.

An embodiment of the invention includes the use of the same top plate with more than one base. An exemplary first base comprises a base with channels or the like adapted and configured to contain growth medium, to provide medium fluid flow with shear forces across the biofilm adherent sites during biofilm growth, and promote growth of the biofilm. An exemplary second base comprises a base adapted and configured with a high number of wells or the like that correspond in number and shape to the adherent sites, e.g., a 96 well microtiter plate.

An embodiment of the invention includes any top being adapted and configured to sealingly engage any base. In these embodiments of the invention, when a top engages a base, the assembly is in a closed, sterile condition. In the most preferred embodiments of the invention, growing the biofilm and assaying the biofilm are conducted under sterile conditions, with the intent of minimizing or eliminating contamination.

In an embodiment of the invention, a top plate or lid may be configured with one or more projections that are sealingly and removably engaged with the plate or lid. In this embodiment of the invention, a projection may be individually removed from the lid without opening or exposing other projections in the lid.

In a preferred method of the invention, the characteristic of the biofilm is the sensitivity of the biofilm to antibacterial reagent and the method further includes, before assaying the biofilm, treating the biofilm adherent sites one or more anti-biofilm agents.

In a further aspect of the invention, after growing the biofilm on adherent sites, dislodging the biofilm from the biofilm adherent sites and further incubating the biofilm. Dislodging the biofilm from the biofilm adherent sites may include dislodging the biofilm from each biofilm adherent site into a separate well of a microtiter plate. In preferred embodiments of the invention, the biofilm is dislodged using any process that results in intact biofilm being removed from the adherent sites. The inventors have found that using centrifugation removes only a portion of the microorganism, and therefore any assay may be incomplete or inaccurate.

Preferably, the plural biofilm adherent sites are formed in plural rows, with plural sites in each row; and the container includes plural channels, with one channel for each row of plural biofilm adherent sites.

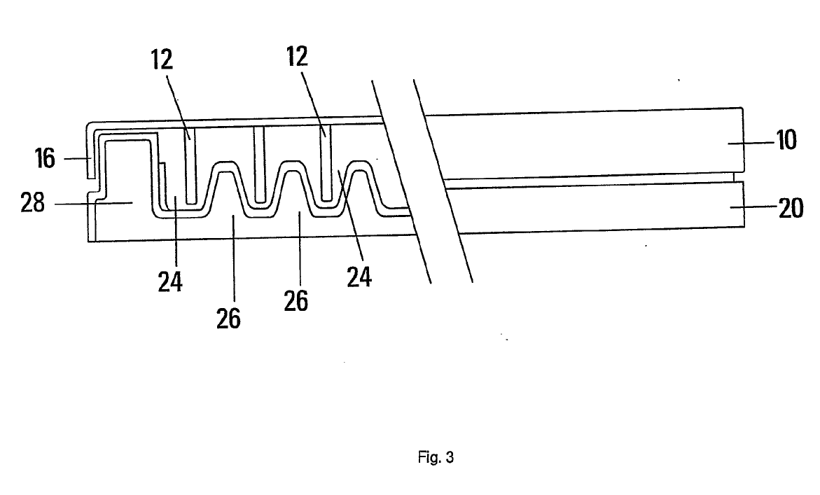

In its entirety, the present invention comprises a biofilm growth assembly 1 and a biofilm assay assembly 2. Used in concert, the two assemblies provide MIC, MBC, and MBEC values in a single experiment. In accordance with the present invention, the biofilm growth assembly 1 may include a base or plate 20 configured to receive a lid 10. Lid 10 may be configured to include one or more projections 12 that extend into a space defined by base 20. In most preferred embodiments of the invention, the biofilm growth assembly 1 is rocked, moved, or the like so that the growth fluid in the assembly flows or moves across projections 12. In preferred embodiments of the invention, base 20 is an incubation base and is configured to provide each projection with substantially equivalent exposure to the source of microorganisms and its nutrient/growth broth. As noted elsewhere in this specification, the typical base includes one of more channels 26. An exemplary configuration is shown in FIG. 3.

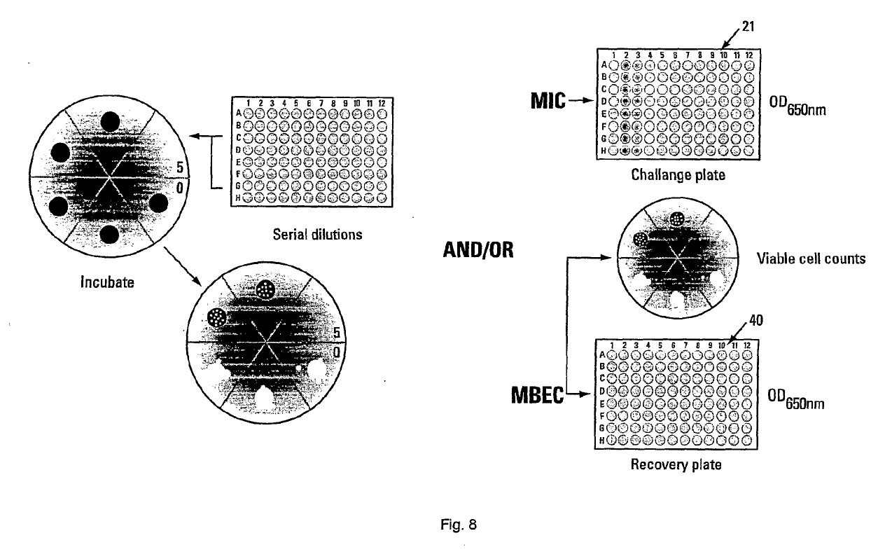

In accordance with the present invention, the biofilm challenge assembly 2 comprises a second base or plate 21 configured to receive a lid 60 having projections 61 typically covered by biofilm. Projections 61 extend into one or more wells configured in plate 21. A typical second base 21 is a standard 96 well microtiter plate, although one skilled in the art will readily recognize that other configurations may be used. Second base 21 includes one or more anti-biofilm agents in the wells. In accordance with the present invention, second plate 21 may be removed and used for determining the MIC value of the non-biofilm (e.g., planktonic) microorganism (see FIG. 8).

Lid 10, with biofilm growing on projections 12 may then be joined with a third base 40 configured to receive the lid 10 and the projections 12. A typical third base 40 is a standard 96 well microtiter plate, although one skilled in the art will readily recognize that other configurations may be used. Third base 40 includes one or more rinsing agents in the wells. After rinsing, lid 10 may then be joined with a fourth base, as used herein, referred to as a recovery plate. The recovery plate contains recovery media, and, in accordance with the present invention, may be subjected to sonication and biofilm re-growth (confirming that the biofilm has not been removed). As shown in the examples, assaying the projections on lid 10 after it has been exposed to recovery media provides an MBEC value of the microorganism, and plating from the recovery plate provides an MBC value. In accordance with the present invention, third base 40 may be removed and used for determining the MIC values of the microorganism.

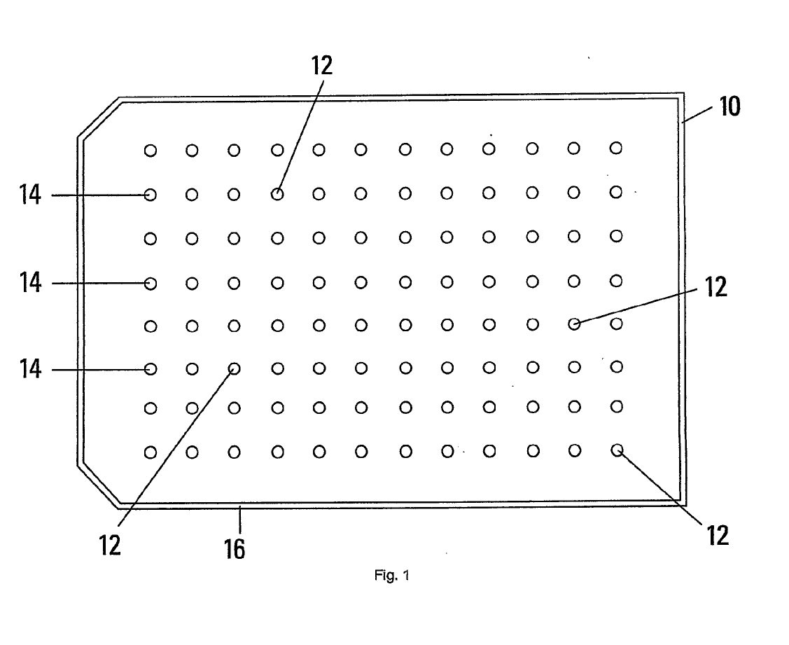

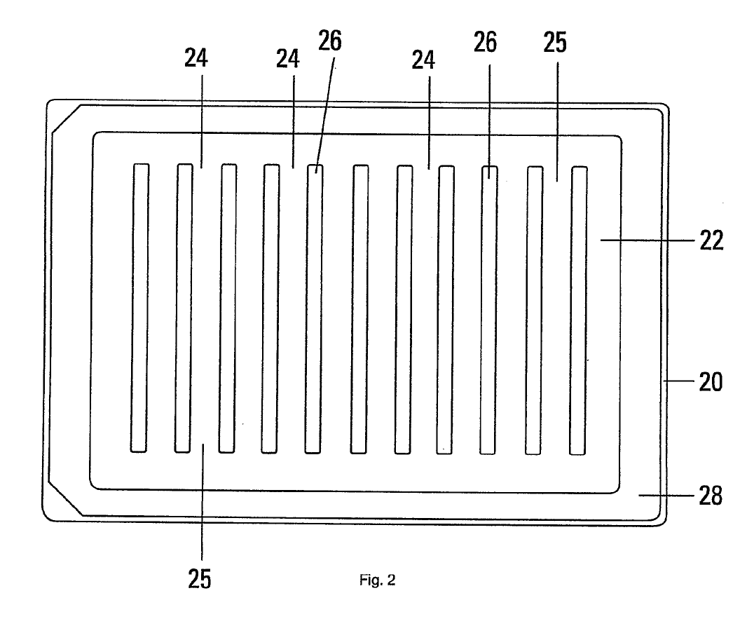

An exemplary embodiment of the invention is described below. As shown most particularly in FIGS. 1, 2 and 3, an exemplary biofilm growth assembly of the present invention includes a lid 10 comprising projections 12, and a base 20 adapted to receive lid 10 and projections 12 and comprising at least one channel 24 or well. As illustrated in the Figures, the deyice includes biofilm lid 10 composed of tissue grade plastic or other suitable material (e.g. stainless steel, titanium) with projections 12 extending downwardly from the lid 10. The projections 12 may be biofilm adherent sites to which a biofilm may adhere, and may be configured into any pattern or shape suitable for use in conjunction with a channel or well-containing bottom, such as base 20. The pattern of projections 12 preferably mirror the pattern of channels and/or wells in convention plates, e.g. a 96 microtiter or well plate commonly used in assay procedures. In most preferred embodiments of the invention, the projections 12 are preferably formed in at least eight rows 14 of at least twelve projections each. Other numbers of rows or numbers of projections in a row maybe used, but this is a convenient number since it matches the 96 well plates commonly used in biomedical devices. Additional projections as shown may be used to determine the initial biofilm concentration after incubation. The exemplary projections 12 shown are about 1.5 cm long and 2 mm wide, but may be any size and/or shape.

The biofilm growth assembly 1 also includes an incubation base 20 configured and adapted to receive lid 10 with projections 12. The lid 10 forms a support for the projections 12 for supporting the biofilm adherent sites within the channels 24. The lid 10 has a surrounding lip 16 that fits tightly over a surrounding wall 28 of the vessel 20 to avoid contamination of the inside of the vessel during incubation.

Base 20 serves two important functions for biofilm development. The first is a reservoir for liquid growth medium containing the bacterial population which will form a biofilin on projections 12. The second function is having a configuration suitable for generating shear force across the projections. While not intending to be limited to any particular theory of operation, the inventors believe that shear force formed by fluid passing across the projections promotes optimal biofilm production and formation on the projections.

Shear force on the projections 12 may be generated by rocking the vessel 20 with lid 10 on a tilt table 30. The inventors have found that using a rocking table that tilts to between about 7° and about 11° is suitable for most applications. In preferred embodiments of the invention, the rocking table should be set on about 9°. It is intended that the invention should not be limited by the use of an actual degree of tilt, but that any tilt used for any particular machine be appropriate for growing biofilm in accordance with the present invention.

The projections 12 may be suspended in the channels 24, so that the tips of the projections 12 may be immersed in liquid growth medium flowing in the channels 24. The ridges 26 channel the liquid growth medium along the channels 24 past and across the projections 12, and thus generate a shear force across the projections. Rocking the vessel 10 causes a repeated change in direction of flow, in this case a repeated reversal of flow of liquid growth medium, across the projections 10, which helps to ensure a biofilm of equal proportion on each of the projections 12 of the lid 10. Rocking the vessel so that liquid flows backward and forward along the channels provides not only an excellent biofilm growth environment, but also simulates naturally occurring conditions.

Each projection 12 and each channel 24 preferably has substantially the same shape (within manufacturing tolerances) to ensure uniformity of shear flow across the projections during biofilm formation. In preferred embodiments of the invention, channels 24 should all be configured or connected so that they share the same liquid nutrient and bacterial mixture filling the basin 22. The inventors have found that substantially uniform channel configuration and access to the same source of microorganisms promotes the production of an equivalent biofilm on each projection, equivalent at least to the extent required for testing anti-biofilm agents. Biofilms thus produced are considered to be uniform. Results have been obtained within P<0.05 for random projections on the plate.

Sensitivity of an anti-biofilm agent may be measured by treating the biofilm adherent sites with an anti-biofilm reagent, and then assaying the biofilm. This may be accomplished by placing the lid 10 (having a biofilm formed on the projections) into a second base 21 adapted to receive lid 10 and projections 12. In preferred embodiments of the invention, lid 10 engages second base 21 in a manner sufficient to prevent contamination of the assembly. As used herein, a manner sufficient to prevent contamination refers to the configuration and assembly of mating structures so that the contents of the closed assembly are free of outside contamination. Projections 12 that have been incubated in the same channel 24 of the vessel 20 should each be treated with a different anti-bacterial reagent. In this manner, consistent results may be obtained since the growth conditions in any one channel will be very similar along the entire channel and thus for each projection 12 suspended in that channel. This helps improves the reliability of treatment of different projections 12 with different anti-bacterial reagents.

Several different conventional methods may be used to count the bacteria. It may be done by incubating the sonicated bacteria, taking serial dilutions and visually counting the colony forming units, or automated methods may be used, as for example using an optical reader to determine optical density. It has been found however that the optical reader of turbidity is too imprecise for practical application, and it is preferred that vital dye technology be applied to automate the measurement of viability, by treating the biofilm with a vital dye, and measuring the intensity of light given off by the dyed biofilm. In the case of using vital dye technology, the biofilm need not be further incubated. In a further embodiment, the assay may be carried out by sonicating the cells until they lyse and release ATP and then adding luciferase to produce a mechanically readable light output. In a still further embodiment, the assay may be carried out directly on the biofilm on the projections using a confocal microscope, although it should be considered that this is difficult to automate. In the examples that follow, the results are obtained from a manual count following serial dilution.

The concentration (MBEC) of anti-bacterial reagent at which the survival of bacteria falls to zero may be assessed readily from the assay. Likewise, the MIC may also be determined from the assay.

The inventors have found that in some instances a biofilm will not form without the inclusion of host components in the biofilm. Host components may therefore be added to the growth medium in the vessel during incubation of the bacteria to form the biofilm. Host components that may be added include serum protein and cells from a host organism. For the testing of the effect of different host cells and components, the ends 25 of the channels 24 may be sealed by walls to prevent growth medium in one channel from flowing into another, thus isolating the bacteria growth in each channel from other channels.

The device thus described may also be used to test coatings used to inhibit biofilm growth and to test coatings which may enhance biofilm formation. In an initial step, the projections 12 may be coated with a coating to be tested, and then the biofilm grown on the projections. The biofilm may then be assayed, or treated with anti-bacterial reagent and then assayed. The assay may be in situ or after dislodging of the biofilm. Different coatings may be tested on different rows of pegs. Enhanced biofilm formation may be used to create large viable biofilms for biofermentation.

As used herein, active agent or anti-biofilm agent refers to one or more agents that are effective in reducing, degrading, or eliminating a biofilm or biofilm-like structures. The present invention includes but is not limited to active agents that are already well known, e.g., antibiotics, anti-microbials, and biocides. One or more active agents may act independently; one or more active agents may act synergistically; one or more active agents may be used sequentially or serially. A composition containing an active agent may further include one or more additional agents, including but not limited to disinfectants, surfactants.

As used herein, biofilm challenge involves the placement of the biofilm culture grown on the pegs MBEC device into the wells of the prepackaged challenge tray such that the patient's isolate is exposed to a range of concentrations of a spectra of antibiotics selected for their complementarity against the target organism. Incubation time and conditions and medium used will vary with isolate.

As used herein, efficacy is based on the ability of the anti-biofilm agent or agents exhibiting activity against the biofilm; and is defined on the basis of MIC (minimal inhibitory concentration), MBC (minimal biocidal concentration), and MBEC (minimal biofilm eradication concentration). The standard assay for testing the antibiotic susceptibility of bacteria is the minimum inhibitory concentration (MIC), which tests the sensitivity of the bacteria in their planktonic phase. The MIC is of limited value in determining the true antibiotic susceptibility of the bacteria in its biofilm phase. The MBEC assay, on the other hand, allows direct determination of the bacteria in the biofilm phase, and involves forming a biofilm in a biofilm growth device or plate, exposing the biofilm to one or more test antibiotics or active agents for a defined period, transferring the biofilm to a second plate having fresh bacteriologic medium, and incubating the biofilm overnight. The MBEC value is the lowest active agent dilution that prevents re-growth of bacteria from the treated biofilm.

As used herein beneficial result refers to any degree of efficacy against a microorganism or biofilm. Examples of benefits include but are not limited to reduction, elimination, eradication, or decrease in a biofilm or a microorganism that forms a biofilm; and the capability of treating a microorganism hidden or protected by a biofilm.

As used herein susceptibility testing or similar phrases refers to determining if and by how much an active agent affects the growth or condition of a microorganism in a biofilm. In the devices and methods of the present invention, susceptibility testing is distinguished from prior art methods by using high through-put devices, and by forming a biofilm in a non-static environment.

As used herein, high throughput refers to the capability of growing and/or assaying a high number of biofilms and/or a high number of anti-biofilm agents at the same time or in the same procedure. Typically, high throughput translates into structural elements in one or more of the assemblies in order to increase speed or quantities of materials being grown or tested, e.g., a 96 well assay plate, a top adapted to and configured to engage the 96 well plate, a top with pegs corresponding to the wells, and a biofilm growth plate with channels so that you can process a large number of individual biofilms at the same time.

### EXAMPLES

The following is used for examples 1-4:

Antibiotic and other antimicrobial stock solutions should be prepared in advance at 5× the highest concentration to be used in the challenge plate. For example, de-ionized water or an appropriate solvent is used to prepare stock solutions of antibiotics at 5120 μg ml⁻¹ of active agent. Consult Clinical Laboratory Standards Institute (CLSI) document M100-S8 for details of which solvents and diluents to use. Stock solutions of most antibiotics are stable for a minimum of 6 months at −70° C.

For research applications it is appropriate to employ a neutralizing agent for determination of minimum bactericidal and fungicidal concentrations. These agents reduce toxicity from the carry-over of biologically active compounds from challenge to recovery media. As examples, it is possible to use β-lactamase to neutralize penicillin, or L-cysteine to neutralize Hg²⁺ and some other heavy metal cations. The following experiments use a universal neutralizer in biocide susceptibility assays that is required for regulatory aspects of product development. This example is presented below:

### 1.0 g L-Histidine

### 1.0 g L-Cysteine

2.0 g Reduced glutathione

Make up to 20 ml in double distilled water. Pass through a syringe with a 0.20 μm filter to sterilize. This solution may be stored at −20° C. Make up 1 liter of the appropriate growth medium (cation adjusted MHB). Supplement this medium with 20.0 g per liter of saponin and 10.0 g per liter of Tween-80. Adjust with dilute NaOH to the correct pH (7.0±0.2 at 20° C.). Add 500 μl of the universal neutralizer to each 20 ml of the surfactant supplemented growth medium used for recovery plates.

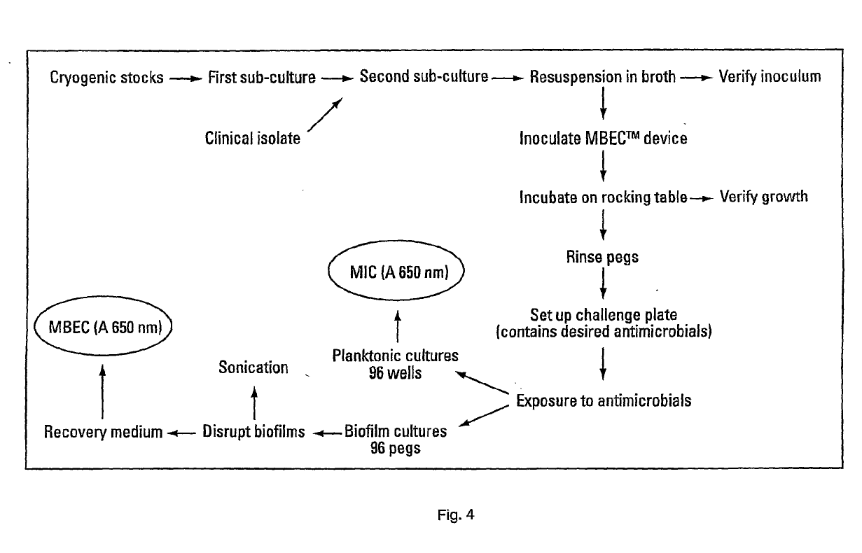

An overview of this experimental protocol is provided in FIG. 4. The number of days required to complete this protocol is dependent on the growth rate of the microorganism being examined. The protocol has been divided into 6 sequential steps, each of which is detailed in the sections below.

This protocol has been developed for use with Nunc Brand, flat bottom, 96-well microtiter plates. These microplates have a maximum volume of 300 μl per well. The medium and buffer volumes listed here may need to be adjusted for different brands of microtiter plates.

### Example 1

### Step 1

### Growing Sub-Cultures of the Desired Microorganism

1. If using a cryogenic stock (at −70° C.), streak out a first sub-culture of the desired bacterial or fungal strain on an appropriate agar plate. Incubate at the optimum growth temperature of the microorganism for an appropriate period of time. For most bacterial strains, the first sub-culture may be wrapped with Parafilm™ and stored at 4° C. for up to 14 days.

2. Check the first sub-culture for purity (ie. only a single colony morphology should be present on the plate).
3. From the first sub-culture or from a clinical isolate, streak out a second sub-culture on an appropriate agar plate. Incubate at the optimum growth temperature of the microorganism for an appropriate period of time. The second sub-culture should be used within 24 h starting from the time it was first removed from incubation.
4. Verify the purity of the second sub-culture.

It is not recommended to grow subcultures on media containing selective agents. Antibiotics and other antimicrobials may trigger an adaptive stress response in bacteria and/or may increase the accumulation of mutants in the population. This may result in an aberrant susceptibility determination.

### Step 2

### Inoculate the Assembly

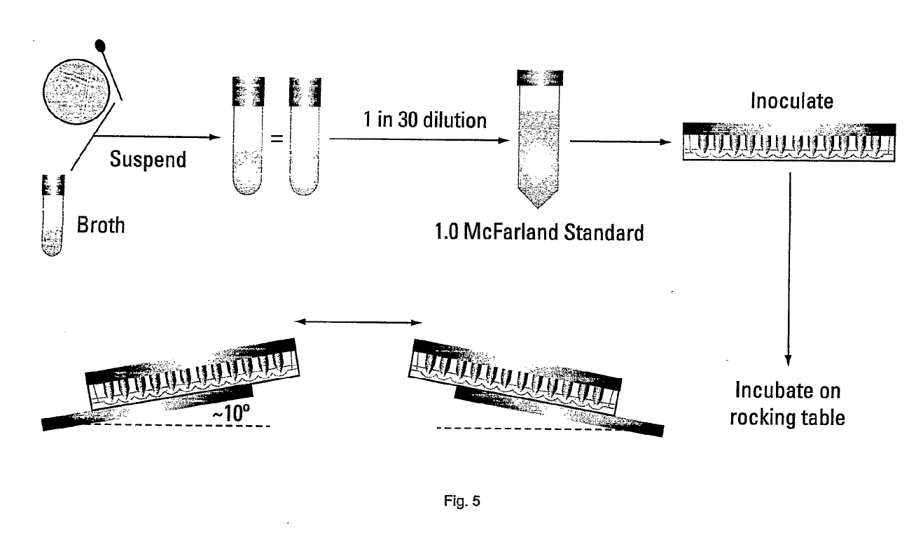

This step, inoculating the assembly, is illustrated in FIG. 5. In summary, a fresh second sub-culture is used to create an inoculum that matches a 1.0 McFarland Standard. This solution is diluted 1 in 30 with growth medium. 22 ml of the 1 in 30 dilution is added to the trough of the base in an assembly of the present invention. The device is placed on a rocking table to assist the formation of biofilms on the polystyrene pegs.

It is recommended that the following steps be carried out in a biological safety cabinet (if available). However, it is possible to use aseptic technique on a bench top:

1. Open a sterile 96-well microtiter plate. For each high throughput assay, fill 4 ‘columns’ of the microtiter plate from ‘rows’ A to F with 180 μl of a physiological saline solution.

2. Put 1.5 ml (plus 1.0 ml for each additional device being inoculated at the same time) of the desired broth growth medium into a sterile glass test tube.

3. Using a sterile cotton swab, collect the bacterial colonies on the surface of the second agar sub-culture. Cover the tip of the cotton swab with a thin layer of bacteria.

4. Dip the cotton swab into the broth to suspend the bacteria. The goal is to create a suspension that matches a 1.0 McFarland standard (ie. 3.0×10⁸ cfu ml⁻¹). Be careful not to get clumps of bacteria in the solution.

5. Repeat step 2, parts 3 and 4 as many times as required to match the optical standard.

6. Put 29 ml of the appropriate broth growth medium (e.g. TSB) into a sterile 50 ml polypropylene or glass tube. To this, add 1.0 ml of the 1.0 McFarland standard bacterial suspension. This 30 fold dilution of the 1.0 McFarland standard (ie. 1.0×10⁷ cfu ml⁻¹) serves as the inoculum for the device.

7. Open the sterile package of the device. Pour the inoculum into a reagent reservoir. Using a sterile pipette, add 22 ml of the inoculum to the trough packaged with the device. Place the peg lid onto the trough.

The volume of inoculum used in this step has been calibrated such that the biofilm covers a surface area that is immersed, entirely, by the volume of antimicrobials used in the challenge plate set up in Step 3 (below). Using a larger volume of inoculum may lead to biofilm formation high on the peg that physically escapes exposure in this challenge step.

8. Place the device on the rocking table in a humidified incubator at the appropriate temperature. The table should be set to between 3 and 5 rocks per minute. It is critical that the angle of the rocking table is set to between 9° and 16° of inclination. This motion must be symmetrical.

9. Serially dilute (ten-fold) a sample of the inoculum (do 3 or 4 replicates). These are controls used to verify the starting cell number in the inoculum.

10. Spot plate the serial 10 fold dilutions of the inoculum from 10⁻⁶ to 10⁻¹ on an appropriately labeled series of agar plates. Incubate the spot plates for an appropriate period of time and score for growth.

### Example 2

### Step 3

### Set Up the Antimicrobial Challenge Plate

The following section describes how to set up a serial two-fold dilution gradient of a single antimicrobial in the challenge plate. This is only one example. The antimicrobial challenge plate may be set up in any manner desired with any combination of antimicrobials. It is important that the final volume in each well of the challenge plate is 200 μl. This is to ensure complete submersion of the biofilm in the antimicrobial.

1. Get a brand new, sterile 96-well microtiter plate and open in it in the laminar flow hood.

2. Setup a working solution of the desired antimicrobial in the appropriate growth medium. Do not dilute the antimicrobial by more than 20% (i.e., no more than 1 part stock antimicrobial solution per 4 parts of growth medium). The working solution of the antimicrobial should be made at a concentration equal to the highest concentration to be tested in the challenge plate.

3. Add 200 μl of growth medium to ‘column’ 1 and ‘column’ 12 of the challenge plate. These will serve as sterility and growth controls, respectively.

4. Add 100 μl of growth medium to ‘columns’ 3 to 11 of the microtiter plate.

5. Add 200 μl of the working solution to ‘column’ 2 of the microtiter plate.

6. Add 100 μl of the working solution to ‘column’ 3 and ‘column’ 4 of the microtiter plate.

7. Using the multi-channel micropipette, mix the contents of ‘column’ 4 by pipetting up and down. After mixing, transfer 100 μl from the wells in ‘column’ 4 to the corresponding wells in ‘column’ 5.

8. Mix and transfer 100 μl from ‘column’ 5 to ‘column’ 6. Serially repeat this mix and transfer process down the length of the microtiter plate until reaching ‘column’ 1.

9. Mix the contents of column 11 up and down. Extract 100 μl from each well in ‘column’ 11 and discard.

10. Add 100 μl of growth media to the wells in ‘columns’ 4 through 11.

11. Replace the lid on the challenge plate. Gently tap the plate to facilitate mixing of biocide/antibiotic and media.

### Step 4

### Expose the Biofilms

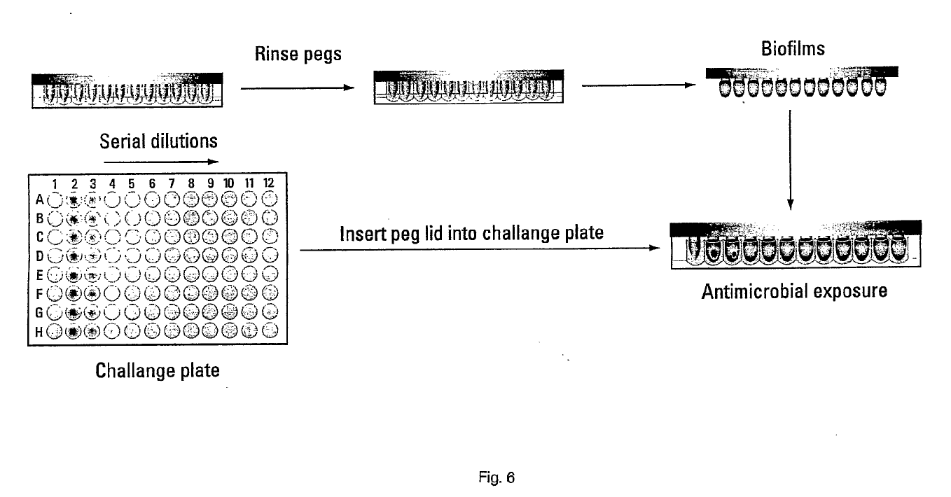

This step, exposing the biofilm to one or more anti-microbials, is illustrated in FIG. 6. In summary, the assembly prepared above is removed from the gyrorotary shaker and the biofilms are rinsed in a physiological saline solution. The rinsed biofilms are then immersed in the antimicrobials of the challenge plate and incubated for the desired exposure time.

1. Setup a sterile microtiter plate with 200 μl of physiological saline solution in every well. This plate will be used to rinse the pegs to remove loosely adherent planktonic cells from the biofilm (this is termed a ‘rinse plate’).

2. This step will be used to determine biofilm growth on four sample pegs and from four wells of the planktonic cultures. Setup a sterile microtiter plate with 200 μl of physiological saline solution in 4 ‘columns’ of row A for each device inoculated (i.e., 1 microtiter plate is required for every 3 devices). Fill rows B to F with 180 μl of physiological saline solution. In a second microtiter plate, fill 4 ‘columns’ from rows A to H with 180 μl of physiological saline solution for each device inoculated. The first microtiter plate will be used to do serial dilutions of biofilm cultures, the second will be used to check the growth of planktonic cells in the wells of the microtiter plate that contained the inoculum.

3. Following the desired period of incubation, remove the high throughput assembly from the rocking table and into the laminar flow hood. Remove the peg lid from the trough and submerse the pegs in the wells of the rinse plate. Let the rinse plate sit for 1 to 2 minutes while performing step 4 below.

4. Use a micropipette to transfer 20 μl of the planktonic culture (in the corrugated trough of the device) into the 180 μl of saline in row ‘A’ of the latter plate set up in step 2 (immediately above). Repeat this three more times for a total of 4×20 oil aliquots.

5. Discard the planktonic culture into the appropriate biohazard waste.

6. In the laminar flow hood, dip a pair of pliers into 95% ethanol. Flame the pliers using the ethanol lamp in the flow hood. Be cautious when using the ethanol lamp. Do not light the ethanol lamp and do not flame the pliers before your hands have dried following disinfection using 70% ethanol.

7. Using the flamed pliers, break off pegs A1, C1, E1 and G1 from the lid of the assembly and immerse them in the 200 μl of saline in row A (and each in a different ‘column’) of the first plate setup in step 2.

8. Using the flamed pliers, break off pegs B1, D1, F1 and H1 and discard.

9. Insert the peg lid of the assembly into the challenge plate. Place the challenge plate in the appropriate incubator for the desired exposure time. Incubations may be carried out at alternative temperatures, taking into consideration extended times for MIC determinations.

10. Place the microtiter plate containing the sample pegs in the tray of the ultrasonic cleaner (the sonicator). Sonicate on the setting ‘high’ for 5 to 30 minutes (the time required depends on the microorganism being assayed). The vibrations created in the water by the sonicator transfer first through the water, then through the steel insert tray, and finally to the device to use vibrations to disrupt biofilms from the surface of the 96 pegs into the saline.

11. Serially dilute 20 μl aliquots of the planktonic cultures (from step 4) in the wells of the corresponding microtiter plate. Once sonication is complete, repeat this serial dilution process with the biofilm cultures.

12. Spot plate the serial 10 fold dilutions of the planktonic and biofilm cultures from 10⁻⁸ to 10⁻³ and 10⁻⁵ to 10⁰ on an appropriately labeled series of agar plates. Incubate the spot plates for an appropriate period of time and score for growth.

### Step 5

### Neutralize and Recover

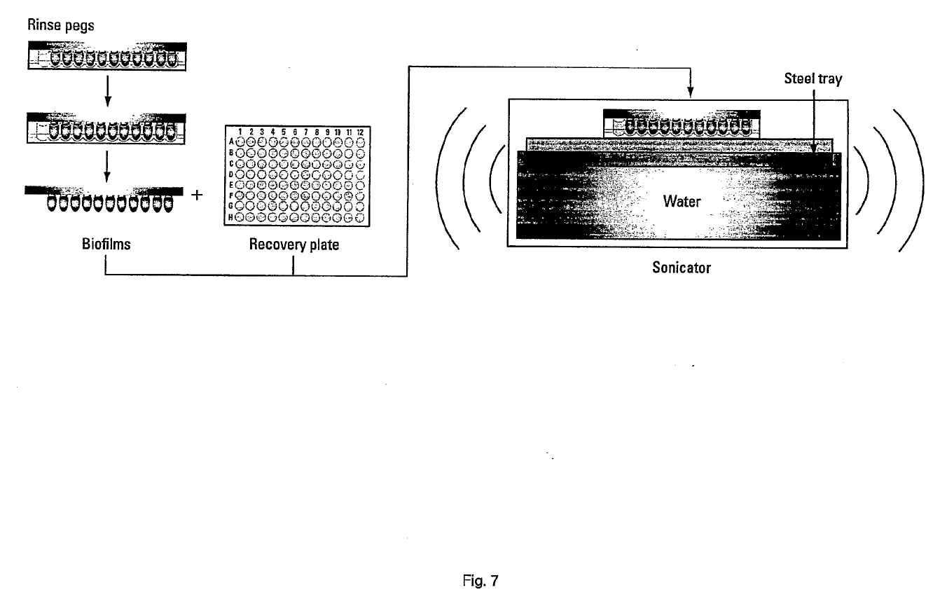

This step, neutralizing the anti-microbials and recovering surviving biofilm bacteria, is illustrated in FIG. 7. In summary, after exposure, biofilms are rinsed twice in physiological saline. The biofilms are then transferred to a microtiter plate containing a neutralizing agent and recovery medium. The biofilms are disrupted into this by sonication on a water table sonicator.

1. Add 200 μl of the appropriate recovery medium (containing a neutralizing agent, see example in section 3.2) to each well of a brand new 96-well microtiter plate. This plate is termed the ‘recovery plate’.

2. Prepare 2 rinse plates for every assembly used.

3. Remove the challenge plate from the incubator and place in the laminar flow hood (or use careful aseptic technique). Remove the peg lid and immerse the pegs in the physiological saline of a rinse plate. Cover the challenge plate with the sterile lid of the rinse plate. After approximately 1 min, transfer the peg lid from the first rinse plate into the second rinse plate. Cover the challenge plate and retain for an MIC determination if appropriate.

4. Transfer the peg lid from the second rinse plate into the recovery plate setup above. Transfer the recovery plate (containing the pegs of the device) onto the tray of the sonicator. Sonicate on high for 5 to 30 min. (depending on the thickness of the biofilm). The vibrations will disrupt biofilms from the surface of the 96 pegs into the recovery plate.

5. After sonication, remove the peg lid from the recovery plate and replace the original lid of the microtiter plate. The lid of the device may now be discarded into autoclave garbage.

6. Place the recovery plate in the incubator and incubate a minimum of 24 to 72 h, depending on the organism being examined.

### Viable Cell Counting

### 5.1 Protocol Amendments

For viable cell counts of biofilms after treatment with an antimicrobial, transfer 100 μl of the recovery media (containing the sonicated biofilms) from the recovery plate to row A of a serial dilution plate. This plate additionally set up to contain 180 μl of physiological saline solution in each well of rows B to F. Serially dilute 20 μl from row A using the multi-channel pipette. Ensure that the tips on the multi-channel pipette are changed between transfers to each row in the microtiter plate. Spot plate biofilm cultures (which have been serially diluted ten-fold) on appropriately labeled agar plates. Incubate for a minimum of 48 hours to ensure maximum recovery of the surviving microorganisms.

Following incubation, enumerate bacteria recovered on plates. Use the formulas in the following section to determine killing of the biofilm population.

To calculate death and survival (log-kill), use the following formula:

log-kill=log 10(initial cfu/ml)−log 10(remaining cfu/ml after exposure)

### Alternatively,

log-kill=log₁₀[1/(1−% kill(as a decimal))]

To calculate percent kill, use the following formula:

% kill=[1−(remaining cfu/ml)/(initial cfu/ml)]×100

To calculate percent survival, use the following formula:

% survival=[(remaining cfu/ml after exposure)/(initial cfu/ml)]×100

To calculate log percent survival, use the following formula:

log % survival=log₁₀(% survival)

### Microscopy

For many microscopy techniques, it may be desirable to fix the biofilms to the surface of the pegs of the assembly. The following protocols may be used to prepare biofilms for scanning electron microscopy (SEM) and confocal laser scanning microscopy (CLSM). In the standard experimental procedure above, each challenge plate has eight growth controls (before exposure). Four of these are used for growth controls. The remaining four may be used for microscopy instead of being discarded.

### Fixing Biofilms for Scanning Electron Microscopy (SEM)

### Preparing Working Solutions

Wear protective gloves in the following steps and handle these highly toxic chemicals in a fume hood.

Cacodylate buffer 0.1 M: dissolve 16 g of cacodylic acid in 1 liter of double distilled H₂O; adjust to pH 7.2.

Glutaraldehyde 2.5% in cacodylate buffer: dissolve 2 ml of 70% glutaraldehyde in 52 ml of cacodylate buffer (yields a 2.5% solution). It is also possible to use a 5% solution (2 ml of glutaraldehyde into 26 ml of cacodylate buffer).

### Standard Protocol

This fixing technique is destructive to biofilms. However, this allows for an examination of the cell structure of the underlying bacteria.

1. Break pegs from the MBEC™-HTP device using a pair of flamed pliers.

2. Rinse pegs in 0.9% saline for 1 min. This disrupts loosely-adherent planktonic bacteria.

3. Fix the pegs in 2.5% glutaraldehyde in 0.1 M cacodylic acid (pH 7.2). Pegs are placed in this solution at 4° C. for 16 h.

4. Following this fixing step, wash the pegs once in 0.1 M cacodylic acid for approximately 10 min.

5. Wash the pegs once in double distilled water for approximately 10 min.

6. Dehydrate the pegs in 70% ethanol for 15 to 20 minutes.

7. Air dry for a minimum of 24 h.

8. Mount specimens and examine by SEM.

### Alternative Protocol

This fixing technique is less destructive. It is possible to observe the extracellular polymeric matrix and some (albeit dehydrated) biofilm structure.

1. Break pegs from the MBEC™-HTP device using a pair of flamed pliers.

2. Rinse pegs in 0.9% saline for 2 min. This disrupts loosely-adherent planktonic bacteria.

3. Fix the pegs in 2.5% glutaraldehyde in 0.1 M cacodylic acid (pH 7.2). Pegs are placed in this solution at 20° C. for 2 to 3 h.

4. Air dry for at least 120 h.

5. Mount specimens and examine by SEM.

### Fixing Biofilms for Confocal Scanning Laser Microscopy (CLSM)

Glutaraldehyde 5% in phosphate buffered saline: dissolve 2 ml of 70% glutaraldehyde in 26 ml of phosphate buffered saline (yields a 5% solution).

Standard protocol

1. Break pegs from the lid using a pair of flamed pliers.

2. Rinse pegs in 0.9% saline for 1 min. This disrupts loosely-adherent planktonic bacteria.

3. Fix the pegs in 5% glutaraldehyde in phosphate buffered saline (pH 7.2). Pegs are placed in this solution at 30° C. for 0.5 to 1 h.

4. Rinse pegs in 0.9% saline for 1 min.

5. Stain pegs with the appropriate fluorphores and examine using the confocal laser scanning microscope.

### Surface Coating the Projections of the Assembly

The surface of the pegs or projections may be coated with a number of materials to facilitate the growth of fastidious microorganisms. For example, biofilm formation by certain Candida spp. is enhanced by coating the pegs with a solution of 1.0% L-lysine. The peg lid may also be coated with hydroxyapetite, collagen, or platinum.

### Example 3

### Determine MBEC Values

To determine the minimum biofilm eradication concentration (MBEC) values, check for turbidity (visually) in the wells of the recovery plate. Alternatively, use a microtiter plate reader to obtain optical density measurements at 650 nm (OD650). Clear wells (OD650<0.1) are evidence of biofilm eradication.

### Example 4

### Determine MIC Values

To determine the minimum inhibitory concentration (MIC) values, check for turbidity (visually) in the wells of the challenge plate. Alternatively, use a microtiter plate reader to obtain optical density measurements at 650 nm (OD₆₅₀). The MIC is defined as the minimum concentration of antibiotic that inhibits growth of the organism. Clear wells (OD₆₅₀<0.1) are evidence of inhibition following a suitable period of incubation.

### Example 5

Background: Pseudomonas aeruginosa (Ps) and Staphylococcus aureus (Staph) form biofilms on tissue and implanted surfaces resulting in persistent infections that are frequently unresponsive to antimicrobial therapy due to biofilm-specific resistance mechanisms. The use of MIC to select antimicrobial therapeutics for biofilm infections is usually not suitable. The MBEC® assay was used for evaluation of antimicrobial susceptibility of biofilm and planktonic bacteria to single and combinations of agents.

Methods: Biofilms of Ps (12 isolates from Cystic Fibrosis patients) and Staph (12 isolates from device associated infections) were formed on the pins of an MBEC® assay lid. Biofilm and Planktonic bacteria were then exposed to various antibiotic and antibiotic combinations for 24 hours (Table 1 and 2). The assay provides qualitative sensitivity of each isolate as a biofilm and planktonic organism to antimicrobial agents alone or in combination.

Results:

TABLE 1
Staph resistance to individual antibiotics
and antibiotic combinations

| Antibiotic | Planktonic | Biofilm |
|---|---|---|
| GM/CLOX | 1 | 12 |
| GM/AMP | 3 | 12 |
| GM/CFZ | 3 | 12 |
| GM/CLIN | 10 | 12 |
| GM/RIF | 12 | 12 |
| GM/CIPRO | 9 | 12 |
| CLIN/RIF | 12 | 12 |
| CLIN/CLOX | 4 | 12 |
| CLIN/VAN | 4 | 12 |
| CLIN/CFZ | 4 | 12 |
| CLIN/CIPRO | 10 | 12 |
| VAN/CFZ | 1 | 12 |
| VAN/CLOX | 3 | 12 |
| VAN/CIPRO | 1 | 12 |
| VAN/GM | 1 | 12 |
| CLIN/AMP | 6 | 12 |
| VAN/RIF | 7 | 11 |
| CIPRO/RIF | 12 | 10 |
| CIPRO/CFZ | 4 | 12 |
| CLOX/RIF | 8 | 12 |
| GM | 6 | 10 |
| AMP | 12 | 12 |
| CFZ | 2 | 12 |
| CIPRO | 10 | 12 |
| VAN | 6 | 12 |
| CLIN | 9 | 12 |
| CLOX | 3 | 12 |

TABLE 2
Ps resistance to individual antibiotics and antibiotic combinations

| Antibiotic | Planktonic | Biofilm |
|---|---|---|
| GM/AZTR | 1 | 12 |
| GM/CFTZ | 3 | 12 |
| TB/AZTR | 1 | 12 |
| TB/CFTZ | 3 | 12 |
| P + T/TB | 1 | 12 |
| P + T/GM | 1 | 12 |
| AK/AZTR | 2 | 12 |
| AK/P + T | 2 | 12 |
| TB/CIPRO | 3 | 12 |
| TB/IMP | 1 | 12 |
| GM/IMP | 8 | 12 |
| CLO/RIF | 8 | 12 |
| AK/CFTZ | 2 | 12 |
| AK/IMP | 4 | 11 |
| CLO/TMS | 0 | 3 |
| CFTZ/AZTR | 11 | 12 |
| CIPRO/AK | 5 | 12 |
| CIPRO/AZTR | 0 | 3 |
| P + T | 4 | 12 |
| CLO | 2 | 12 |
| AZTR | 0 | 9 |
| CIPRO | 4 | 12 |
| GM | 8 | 12 |
| AK | 8 | 11 |
| TB | 3 | 12 |
| TMS | 1 | 6 |
| CFTZ | 3 | 12 |
| IMP | 12 | 12 |

Conclusions: The resistance patterns were unique to each strain. Ps and Staph strains were sensitive to multiple antibiotics as planktonic forms but significantly more resistant as a biofilm. Certain antibiotics were more effective as combinations than as individual agents. The MBEC® assay may be useful in the selection of antibiotics for treatment of biofilm associated infections.

### Example 6

### bioFILM PA Antimicrobial Susceptibility System

bioFILM PA panels are designed for use in determining antimicrobial agent susceptibility of both planktonic and biofilm Pseudomonas aeruginosa.

### Summary and Principles

This broth dilution antimicrobial susceptibility test has various antimicrobial agents alone and in combination which are diluted in cation adjusted Mueller-Hinton broth (CAMHB) at categorical breakpoint concentrations defined by Clinical and Laboratory Standard Institute™ (CLSI). Panel wells are inoculated with planktonic and biofilm Pseudomonas aeruginosa using a 95 peg inoculation lid. Panels and pegged lids are then incubated at 35° C. for a minimum of 16 hours. Planktonic susceptibility and resistance is determined by measuring inhibition and growth in the presence of antimicrobial agents after 16-24 hours incubation at 35° C. The pegged lid containing the biofilm bacteria that have been exposed to the antimicrobial agents is placed in a recovery media containing only CAMHB in 96 well plate. Biofilm susceptibility and resistance is determined by measuring inhibition and growth after incubation for additional 16-24 hours at 35° C.

### Procedure Materials

bioFILM PA Breakpoint Panel
Sterile recovery panel with lid
Sterile MBECTM 95 pegged inoculation lid
Sterile MBECTM tray (for growth of inoculum)
Sterile rinse panel

### 0.5 McFarland Barium Sulfate Turbidity Standard

Inoculum water

### Inoculum Broth (Tryptic Soy Broth)

Test and recovery broth, Cation Adjusted Mueller Hinton Broth (CAMHB)
100 μl pipette with disposable tips
Multichannel micropipettes (50-300 μl with 12 channels recommended)
Rocking Platform (9° tilt angle)

### Incubator

25 ml pipette
Sterile 50 ml tube with screw top
Quality control organism (Pseudomonas aeruginosa, ATCC 27853)
Quality control report forms
96 well microtiter plates (1 for peg washing and 1 for recovery plate)

### Vortex

### Procedure Outline

### A. Inoculum Preparation

CLSI recommends periodically checking inoculum densities by doing colony counts. The expected results for Pseudomonas aeruginosa ATCC 27853 should closely approximate 5×10⁵ CFU/ml^{2,3}.

### 1. Primary Inoculation Method

The turbidity standard technique is recommended for direct inoculation of Pseudomonas aeruginosa.
a. Using a sterile wooden applicator stick or bacteriological loop, touch the surface of 4-5 large or 5-10 small morphologically similar, well-isolated colonies from an 18-24 hour noninhibitory agar plate.
b. Emulsify in 3 ml of Inoculum Water (autoclaved distilled water) and compare with 0.5 McFarland standard.
c. Vortex the suspension for 2-3 seconds.
d. Pipette 88 g1 of the standardized suspension into sterile 22 ml of Tryptic Soy Broth (TSB). Cap tightly. Invert 8-10 times or vortex sample for few seconds to mix
   2. Preparation of bioFILM PA Inoculation Peg Lid
a. Remove MBECTM tray and 95 pegged lid from the package (Do not use if integrity of the packaging is compromised).
b. Remove pegged lid from tray.
c. Pipette 22 ml of Pseudoinonas aeruginosa suspension (see Id) in TSB to slotted tray.
d. Place 95 pegged inoculation lid on the tray (check that the pegged lid is properly aligned to fit securely over the tray)
e. Place the assembled pegged lid and tray on rocking platform with approximately 9° tilt. Align the troughs parallel to the direction of rocking.
f. Incubate at 35° C. with 3-4 rocks per minute. Some rockers are not suitable for the bioFILM PA assay as the tilt angle is too great or the platform rocking is asymmetrical. The operator should check to see that the rocker meets the necessary requirements for this assay.
f. Incubate for 4-6 hours. This is sufficient to generate a biofilm of approximately 10⁵ cfu/peg.

### B. Panel Preparation

1. Remove the panels to be used from frozen storage. Cut open the pouch and remove the panel and allow to equilibrate to room temperature for 30 to 60 minutes.

### C. Planktonic Antimicrobial Sensitivity Testing

1. Place prepared MBECTM 95 pegged lid on thawed bioFILM PA panel.
2. A final well concentration of planktonic Pseudoinonas aeruginosa of 3-7×10⁵ CFU/ml should be achieved².
3. A purity plate should be prepared by streaking the inoculum on blood agar plate and incubate for 16-20 hours. If more than one colony morphology is present on the purity plate, re-isolation of test colonies and retesting of the panel is warranted.

### D. Antimicrobial Panel Incubation

1. Stack the panels in groups of 3-5 to ensure even thermal distribution during incubation.
2. Incubate the panels for 16-24 hours at 35° C. in a non-CO₂ incubator.
3. After incubation for 16-24 hours the Planktonic panel is ready to be read.
4. Remove pegged lid (see/follow section E 1.)
5. Read planktonic antimicrobial sensitivity results (see below F).

### E. Biofilm Antimicrobial Sensitivity Testing

1. Place pegged lid in 96 well plate containing 200 gl±10 gl per well of Inoculum Water for approximately 30 seconds. This is performed to remove any residual antibiotics from pegs.
2. Place pegged lid in 96 well recovery plate containing 200 gl of CAMHB in each well.
3. To ensure even thermal distribution during incubation, stack the panels in groups of no more than 5.
4. Incubate the panels for 16-24 hours at 35° C. in a non-CO₂ incubator.
5. After incubation for 16-24 hours the Biofilm recovery panel is ready to be read.
6. Remove pegged lid and discard.
7. Read Biofilm antimicrobial sensitivity results (see below F).

### F Reading the Panels for Planktonic and Biofilm Susceptibility

1. Remove the panels after 16-20 hours incubation.
2. Wipe off the bottom of the panel with a lint-free tissue to remove any condensation or debris that may be present.
3. Growth in the antimicrobial wells appears as turbidity, which may take the form of a white haze throughout the well, a white button in the center of the well, or a fine granular growth throughout the well. Inadequate or no growth is defined as a slight whiteness in the well or the broth.
4. Precautions for reading the panels:
   a. Only read the panels if the growth well is turbid.
   b. Do not read the antimicrobial sensitivity wells if the Sterility Control well (SC) is turbid, or if there is no growth in the growth well or Growth Control (GC).

### G. Reading Planktonic Antimicrobial Susceptibilities:

| Description | Conclusion | Code |
|---|---|---|
| Following 16-20 hours incubation, no growth | Susceptible | S |
| in the antimicrobial higher and lower | | |
| concentration of antimicrobial agent | | |
| Growth in the lower concentration of the | Intermediate | I |
| antimicrobial agent, but not in the higher | | |
| concentration. | | |
| Growth in both concentrations of the | Resistant | R |
| antimicrobial agent. | | |

### Interpretation of Results

Susceptibility is determined by comparing the breakpoint susceptibility of an organism with either the attainable blood or urine level of the antimicrobial agent. The following table lists the interpretive criteria as indicated in the CLSI document M100-S9

J. Interpretive Breakpoints*

| Antimicrobial Agent | Susceptible | Intermediate | Resistant |
|---|---|---|---|
| Amikacin | <16 | 32 | >64 |
| Aztreonam | <8 | 16 | >32 |
| Cefepime | <8 | 16 | >_32 |
| Ceftazidime | <8 | 16 | >32 |
| Chloramphenicol | <8 | 16 | >32 |
| Ciprofloxacin | <1 | 2 | >4 |
| Colistin | <2 | — | >4 |
| Gentamicin | <4 | 8 | >16 |
| Meropenem | <4 | 8 | >_16 |
| Piperacillin/tazobactam | <16/4 | 32/4-64/4 | >128/4 |
| Trimethoprim/sulfamethoxazole | <2/38 | — | >4/76 |
| Tobramycin | <4 | 8 | >16 |
| Amikacin/aztreonam | <16/8 | 32/16 | >64/32 |
| Amikacin/cefepime | <16/8 | 32/16 | >64/32 |
| Amikacin/ceftazidime | <16/8 | 32/16 | >64/32 |
| Amikacin/ciprofloxacin | <16/1 | 32/2 | >64/4 |
| Amikacin/colistin | <16/2 | — | >64/4 |
| Amikacin/meropenem | <16/4 | 32/8 | >64/16 |
| Amikacin/piperacillin/tazobactam | <16/16/4 | 32/32/4-32/64/4 | >64/128/4 |
| Amikacin/trimethoprim/sulfamethoxazole | <16/2/38 | — | >64/4/76 |
| Chloramphenicol/ceftazidime | <8/8 | 16/16 | >32/32 |
| Chloramphenicol/meropenem | <8/4 | 16/8 | >32/16 |
| Ciprofloxacin/aztreonam | <1/8 | 2/16 | >4/32 |
| Ciprofloxacin/colistin | <1/2 | — | >4/4 |
| Ciprofloxacin/meropenem | <1/4 | 2/8 | >4/16 |
| Ciprofloxacin/piperacillin/tazobactam | <1/16/4 | 2/32/4-2/64/4 | >4/128/4 |
| Ciprofloxacin/trimethoprim/sulfamethoxazole | <1/2/38 | — | >4/4/76 |
| Gentamicin/aztreonam | <4/8 | 8/16 | >16/32 |
| Gentamicin/cefepime | <4/8 | 8/16 | >16/32 |
| Gentamicin/ceftazidime | <4/8 | 8/16 | >16/32 |
| Gentamicin/ciprofloxacin | <4/1 | 8/2 | >16/4 |
| Gentamicin/colistin | <4/2 | — | >16/4 |
| Gentamicin/meropenem | <4/4 | 8/8 | >16/16 |
| Gentamicin/piperacillin/tazobactam | <4/16/4 | 8/32/4-8/64/4 | >16/128/4 |
| Gentamicin/trimethoprim/sulfamethoxazole | <4/2/38 | — | >16/4/76 |
| Tobramycin/aztreonam | <4/16 | 8/32 | >16/64 |
| Tobramycin/cefepime | <4/8 | 8/16 | >16/32 |
| Tobrainycin/ceftazidime | <4/8 | 8/16 | >16/32 |
| Tobramycin/ciprofloxacin | <4/1 | 8/2 | >16/4 |
| Tobramycin/colistin | <4/2 | — | >16/4 |
| Tobramycin/meropenem | <4/4 | 8/8 | >16/16 |
| Tobramycin/piperacillin/tazobactam | <4/16/4 | 8/32/4-8/64/4 | >16/128/4 |
| Tobramycin/trimethoprim/sulfamethoxazole | <4/2/38 | — | >16/4/76 |
| Trimethoprim/sulfamethoxazole/aztreonam | <2/38/16 | — | >4/76/64 |
| Trimethoprim/sulfamethoxazole/ceftazidime | <2/38/8 | — | >4/76/32 |
| Trimethoprim/sulfamethoxazole/meropenem | _<2/38/4 | — | >_4/76/16 |
| Trimethoprim/sulfamethoxazole/piperacillin/* | <2/38/16/4 | — | >4/76/128/4 |

Based on Interpretive Breakpoints as indicated in CLSI Document M100-S16

### REFERENCES

1. Murray, P R., E. J. Baron, M. A. Pfaller, F. C. Tenover and R. H. Yolken (eds) 2003. Manual of Clinical Microbiology, 8th Ed. American Society of Microbiology Washington, D.C.
2. Clinical Standard Laboratory Institute TM (2006). Methods for dilution antimicrobial susceptibility tests for bacteria that grow aerobically; 6th ed. Approved standard M7-A7.
3. Clinical Standard Laboratory Institute™ (2006). Performance Standards For Antimicrobial Susceptibility Testing; 16th informational supplement, Wayne, Pa. CLSI document M100-S16, Clinical and Laboratory Standards Institute, 940 West Road Suite 1400, Wayne, Pa.

### Example 7

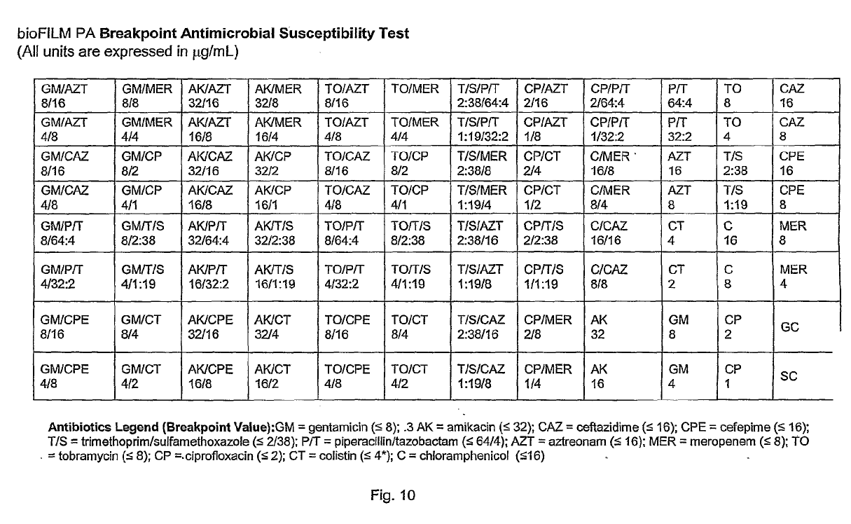

The experiment described in Example 6 was repeated using a challenge plate configuration and breakpoints shown in FIG. 10.

### Example 8

In this study, a Pseudomo as aeruginosa biofilm assay kit was used to test the effect of 10 antibiotics and combinations of these antibiotics at different concentrations, and to compare the effects of antibiotics on two strains of P. aeruginosa, CF 6649 and CF 6106.

Antibiotic and antibiotic combinations were selected based on the results of preliminary studies that demonstrated effectiveness among antibiotic combinations to microbial biofilms.
Forming the biofilm: A suspension of the organism such that the turbidity matches a McFarland standard of 1.0 (approx. 3.0×108 cfu/mL) in TSB was prepared. A 30 mL inoculum was prepared by diluting the suspension 1/30 for an initial inoculum of 107 cfu/mL. 22 mL of the inoculum was placed into a trough of an assembly of the present invention, and the peg lid was replaced. The device was placed on a rocking platform at 35° C., approx. 9° tilt, and 3-4 rocks per minute, with the troughs parallel to the direction of rocking. The target was to generate a biofilm of >105 cfu/peg; this was achieved in less than 24 hour incubation.

A 96-well tissue culture plate was used to prepare the challenge plate. 20 μL of each test antibiotic was placed in the 96-well tissue culture plate and 180 μL of Cation Adjusted Mueller Hinton Broth (CAMHB) to was added to each well of the microtiter plate to achieve a 1:10 dilution of test drug. Two wells (G12 & H12) were empty or included 200 μL of Sterile Normal Saline. G12 and H12 served as Sterility Control. Similarly, A12 & B12 served as Growth Control.

The lid with the pegs were placed on the challenge plate and incubated at 35° C. for 24 hours.

A rinse plate(s) of saline (200 μL per well) in a sterile 96 well microtiter plate was prepared. A recovery plate(s) of CAMHB (200 μL per well) in another 96 well microtiter plate was also prepared. Pegs were placed in saline. Pegs were transferred to recovery media, and then sonicated on high for 5 minutes to dislodge surviving biofilm. The pegs were then incubate at 35° C. for 20 to 24 hours to allow surviving bacteria to grow to turbidity.

Planktonic MIC was determined by visually checking turbidity in the wells of the challenge plate and on a plate reader at 650 nm. The MIC (minimum inhibitory concentration) for each antibiotic for the planktonic bacteria shed from the biofilm during the challenge incubation was determined. The MIC is defined as the minimum concentration of antibiotic that inhibits growth of the organism. Clear wells (A₆₅₀<0.1) are evidence of inhibition.

Biofilm MBEC (minimum biofilm elimination concentration) was determined for each antibiotic by reading the turbidity of the recovery plate. The MBEC is defined as the minimum concentration of antibiotic that inhibits re-growth of the biofilm bacteria in the recovery media. Clear wells (A₆₅₀<0.1) are evidence of inhibition.

Pseudomonas aeruginosa MBEC Test plate: GM=gentamicin, AK=amikacin, CFTZ=ceftazidime, TMS=trimethoprim/sulfamethoxazole, P+T=piperacillin/tazobactam (concentration given as the piperacillin), AZTR=aztreonam, IMP=imipenem, TB=tobramycin, CIPRO=ciprofloxacin, GC=growth control, SC=sterility control.

The sensitivity of planktonic and biofilm forms of P. aeruginosa to individual and combination antimicrobial agents can be determined rapidly (48 hours) and reproducibly (Table 1). The resistance patterns were unique for each isolate. P. aeruginosa was sensitive to multiple antibiotics as planktonic forms but significantly more resistant as a biofilm.

TABLE 1
Number of P. aeruginosa isolates resistant to individual
antibiotics and antibiotic combinations

| Antibiotic | Planktonic | Biofilm |
|---|---|---|
| GEN/ATM | 1 | 12 |
| GEN/CAZ | 3 | 12 |
| TOB/ATM | 1 | 12 |
| TOB/CAZ | 3 | 12 |
| TZP/TOB | 1 | 12 |
| TZP/GEN | 1 | 12 |
| AK/ATM | 2 | 12 |
| AK/TZP | 2 | 12 |
| TOB/CIP | 3 | 12 |
| TOB/IPM | 1 | 12 |
| GEN/IPM | 8 | 12 |
| CT/RA | 8 | 12 |
| AK/CAZ | 2 | 12 |
| AK/IPM | 4 | 11 |
| CT/SXT | 0 | 3 |
| CAZ/ATM | 11 | 12 |
| CIP/AK | 5 | 12 |
| CIP/ATM | 0 | 3 |
| TZP | 4 | 12 |
| CT | 2 | 12 |
| ATM | 0 | 9 |
| CIP | 4 | 12 |
| GEN | 8 | 12 |
| AK | 8 | 11 |
| TOB | 3 | 12 |
| SXT | 1 | 6 |
| CAZ | 3 | 12 |
| IPM | 12 | 12 |

Certain antibiotics were more effective as combinations than as individual agents. The assay offers the clinician 10 single and 82 combinations of antibiotics at breakpoint concentrations. This assay may be useful for clinicians in the selection of antibiotics for treatment of biofilm associated infections that are common in cystic fibrosis patients.

This experiment demonstrates that each strain has unique biofilm sensitivity. This is surprising because the planktonic data is very similar among isolates. These results clearly indicate that organisms have different sensitivity toward individual antibiotic or combinations of antibiotics, depending on their growth condition (planktonic or biofilm).

### Example 9

### Staph Test Plate Assembly

A prototype Staphylococcus test plate was developed to evaluate antibiotics and antibiotic combinations that can be used to treat Staphylococcus infections. The antibiotic and antibiotic combinations selected are based on the results of preliminary studies that demonstrated effectiveness among antibiotic combinations to microbial biofilms. The prototypes 96 well plate is described below: Staphylococcus Test plate: GM=gentamicin; CLIN=clindamycin; CFZ=cefazolin; CLOX=cloxacillin; RIF=rifampin; VAN=vancomycin; LIZD=Linezolid; AMP=ampicillin sublactamj; Cipro=Ciprofloxacin; GC=growth control; SC=sterility control

The sensitivity of planktonic and biofilm forms of Staphylococcus aureus to individual and combination antimicrobial agents can be determined rapidly (within about 48 hours) and reproducibly (Table 2). The resistance patterns were unique for each isolate. Staphylococcus aureus was sensitive to multiple antibiotics and antibiotic combinations as planktonic forms, but significantly more resistant as a biofilm.

TABLE 2
Number of Staphylococcus aureus isolates resistant to
individual antibiotics and antibiotic combinations

| Antibiotic | Planktonic | Biofilm |
|---|---|---|
| GEN2/CLO | 2 | 10 |
| GEN4/CLO | 0 | 10 |
| GEN2/AMP | 0 | 09 |
| GEN4/AMP | 1 | 12 |
| GEN2/CFZ | 3 | 09 |
| GEN4/CFZ | 1 | 10 |
| GEN2/CLN | 5 | 12 |
| GEN4/CLN | 7 | 12 |
| GEN2/RIF | 11 | 09 |
| GEN4/RIF | 11 | 10 |
| GEN2/CIP | 4 | 11 |
| GEN4/CIP | 2 | 11 |
| CLIN1/RIF | 10 | 12 |
| CLIN2/RIF | 10 | 12 |
| CLIN1/CLO | 2 | 12 |
| CLIN2/CLO | 2 | 12 |
| CLIN1/VAN | 1 | 12 |
| CLIN2/VAN | 0 | 12 |
| CLIN1/CFZ | 4 | 12 |
| CLIN2/CFZ | 2 | 12 |
| GM2 | 4 | 11 |
| GM4 | 3 | 10 |
| AMP4/2 | 10 | 12 |
| AMP8/4 | 10 | 12 |
| CFZ4 | 4 | 12 |
| CFZ8 | 6 | 12 |
| CIP1 | 7 | 12 |
| CLIN1/CIP | 9 | 12 |
| CLIN2/CIP | 10 | 12 |
| VAN2/CFZ | 0 | 12 |
| VAN4/CFZ | 1 | 12 |
| VAN2/CLO | 0 | 12 |
| VAN4/CLO | 0 | 12 |
| VAN2/CIP | 1 | 12 |
| VAN4/CIP | 0 | 12 |
| VAN2/GM | 0 | 09 |
| VAN4/GM | 0 | 08 |
| CLI1/AM | 4 | 12 |
| CLI2/AM | 2 | 12 |
| VAN4/RIF | 7 | 10 |
| VAN8/RIF | 7 | 11 |
| CIP2/CFZ | 3 | 09 |
| CIP4/CFZ | 7 | 10 |
| CIP2/RIF | 4 | 12 |
| CIP4/RIF | 3 | 11 |
| CLO2/RIF | 5 | 12 |
| CLO4/RIF | 4 | 12 |
| CIP2 | 7 | 12 |
| VAN2 | 5 | 12 |
| VAN4 | 1 | 12 |
| CLI1 | 8 | 12 |
| CLI4 | 5 | 12 |
| CLO2 | 2 | 12 |
| CLO4 | 2 | 12 |

Certain antibiotics were more effective as combinations than as individual agents. The assay offers the clinician 10 single and 82 combinations of antibiotics at breakpoint concentrations.

This experiment demonstrates that each strain has unique biofilm sensitivity. This is surprising because the planktonic data is very similar among isolates. These results clearly indicate that organisms have different sensitivity towards individual antibiotic or combinations of antibiotics depending on their growth condition (planktonic or biofilm).

### Example 10

### Comparative Susceptibility of Planktonic and Biofilm Forms of

Candida spp. and Aspergillus fumigatus to Antifungal Agents

Three clinical isolates of Candida were used in this study, including one albicans and two non-albicans species. C. albicans ATCC 14053 was obtained from the University Of Calgary, Department Of Biological Sciences. C. tropicalis 99916 and C. glabrata 14326 were obtained from the dialysate of patients undergoing continued ambulatory peritoneal dialysis (CAPD). Aspergillus fumigatus was also tested.

Biofilm formation and measurement of antimicrobial sensitivity of Candida and Aspergillus biofilms were performed using an assembly of the present invention. The device features a microtliter plate lid with 96 pegs or projections distributed on the lid. Each peg provided the surface for microorganism to adhere, colonize and form a uniform biofilm. The pegs fit precisely into the wells of a standard 96-well microtiter plate. The lid was used in conjunction with a base having special troughs for growing, washing, and incubating fungi. Colonies of Candida sp. were picked from 24 hour cultures on Sabouraud Dextrose agar (SDA) (BBL Microbiological Systems, Cockeysville, Md.) and placed into Mueller-Hinton Broth (MHB) (Difco Laboratories, Detroit Mich.) such that the suspension matched a McFarland standard of 1.0. This suspension was then diluted 1:30 in MHB, and 25 ml were pipetted into the trough chamber of the base. The closed assembly (lid with pegs positioned on the base) was placed on a Hoeffer Red Rocker (VWR, Scientific) set to rock the plates 3.5 cycles per minute in a 37° C. incubator. C. albicans and C. tropicalis were incubated aerobically, while C. glabrata was incubated in 10% CO₂. It was determined in preliminary experiments that C. glabrata required 10% CO₂ atmosphere for biofilms to form.

The growth curves were obtained for each isolate by randomly removing 3 pegs from the lid of the device at 1, 2, 3, 4, 5, 6, 7, 22, 23, 24 and 26 hours post-inoculation. The removed pegs were placed in microfuge tubes containing 200 μl of saline, and sonicated (Aquasonic sonicator, VWR Scientific,) for 5 minutes. Serial dilutions were performed and plate counts of viable Candida spp. cells were performed on SDA. Additional pegs containing 22 hour Candida spp. biofilms were fixed with 2.5% glutaraldehyde in phosphate buffered saline solution (PBSS), air-dried overnight, and prepared for scanning electron microscopy.

Optimal conditions for the formation of A. fumigatus biofilms were determined in preliminary studies. The pegs were first soaked overnight in 1% L-lysine (Sigma Chemical Co, St. Louis, Mo.) and then air-dried inside a laminar flow hood. A 50 μl volume of A. fumigatus spore suspension was added to 250 ml of Tryptic Soy Broth (TSB) (Difco, Detroit, Mich.) in a 500 ml Erlynmeyer flask. The flask was shaken at 150 rpm for 20 hours at 37° C. The adherent mycelial cells growing on the glass at the apex of the liquid broth were removed with sterile cotton swab. This was transferred to a blender (Waring) with 250 ml of fresh TSB equilibrated to 4° C. The mycelium was blended at medium speed over ice for two minutes. This was repeated three times. The A. fumigatus suspension (25 ml) was then transferred to the trough of the biofilm growth device. The lid containing the lysine-treated pegs was placed in the suspension and the device was transferred to a Red Rocker as described above. The plates were incubated for 25 hours at 37° C., establishing a visible biofilm on each peg. Scanning electron microscopy examination of pegs containing 24 hour Aspergillus biofilms was performed.

### Minimum Biofilm Eradication Concentration Assay

Biofilm susceptibility testing uses the pegged lid of the assembly, now containing biofilms formed after rocking in the tray for 24 hour. Each peg on the lid was gently washed once in 2001 of phosphate buffered saline solution (PBSS) in a 96-well microtiter plate (Falcon). The pegged lid was then transferred to another 96 well microtiter plate containing 2 fold dilutions antifungal agent in 200 μl of RPMI 1640 (Sigma, St. Louis, Mo.) or RPMI 1640 containing 1% DMSO (see test drug section). After the pegs were exposed to the drugs for 24 hours, the pegged lid was removed and gently rinsed twice in saline. The pegged lid was then placed on a 96 well plate containing RPMI 1640 recovery medium. The pegs were sonicated for 5 minutes (Candida spp.) or 7 minutes (Aspergillus) in an ultrasonicator to dislodge adherent cells into the recovery medium. Aliquots of 20 μl of the recovery medium were spot plated on SDA (Candida spp) or Rose Bengal Agar (A. fumigatus) to obtain the MBEC. The assembly was also used to determine the minimum inhibitory concentration (MIC) and minimum fungicidal concentration (MFC). The turbidity of the wells that contained the antibiotic and planktonic cells which were shed from the biofilm was measured at 650 μm to obtain the MIC. A 20 μl sample from each well was also spot plated onto Sabouraud Dextrose agar (Candida spp) or Rose Bengal Agar (A. fumigatus) to obtain the MFC.

Planktonic Sensitivity of Candida spp. and A. fumigatus:

Minimum Inhibitory Concentrations (MIC) and Minimum Fungicidal Concentrations (MFC) were determined according to the guidelines of the National Committee for Clinical laboratory Standards (NCCLS). Briefly, Candida sp. were maintained on polystyrene spheres at −70° C. until plating. These organisms were streaked onto Sabouraud Dextrose agar (Microbiologie) plates 24 hours before test initiation. The 24 hour colonies were picked from the agar plates and placed in Muellar-Hinton Broth (MHB, Microbiologie) such that the turbidity matched a McFarland standard of 0.5. This was then diluted 1:10 in MHB to obtain a suspension of approximately 1×10⁵ to 5×10⁵ CFU/ml. A 5 μl volume of this suspension was added to wells containing 200 μl of antifungal agent serially diluted in RPMI 1640 media (Sigma) in a 96 well microtiter plate (Nunclon). A. fumigatus was maintained as a spore suspension at 4° C. until testing. The spore suspension (50 μl) was diluted into 250 ml of TSB, and 5 μl of this suspension was added to each test well containing dilution of antifungal agent as above.

The test wells were then incubated at 37° C. for 24 hours with the antifungal drugs. MICs for Candida were obtained after incubation by reading the turbidity at 650 nm on a microtiter plate reader (Softmax, VWR). A 20 μl aliquot of each well was also plated (SDA) and MFCs obtained from them after 24 hours incubation at 37° C. MFCs were determined for Aspergillus by plating 100 μl from each well onto Rose Bengal Agar, followed by spreading with a sterile glass spreader. The plated organisms were maintained at 25° C. for three days before colony enumeration.

Drugs tested. Itraconazole, Fluconazole, and Ketoconazole were obtained from Janssen Pharmaceuticals in Brussels, Belgium. Nystatin 5-Fluorocytosine, Griseofulvin, Amphotericin B and Polymyxin B were obtained from Sigma Chemical Co, St. Louis, Mo. 5-Fluorocytosine, polymyxin B sulphate and nystatin were dissolved in double-distilled water to a concentration of 10.24 mg/ml and diluted in the test wells through a range of 1024 μg/ml to 0.125 μg/ml in RPMI. Amphotericin B was dissolved in neat dimethylsulfoxone (DMSO) to a concentration maximum of 51.2 mg/ml. This was serially diluted to achieve 512 μg/ml to 0.016 μg/ml of Amphotericin B in RPMI 1640 (Sigma, St. Louis Mo.) and 1% DMSO in the test wells. Fluconazole, itraconazole, ketoconazole and griseofulvin were dissolved in neat DMSO to a concentration of 102.4 mg/ml and further diluted to achieve a range range of 1024 μg/ml to 0.032 μg/ml drug in RPMI 1640 and 1% DMSO in the test wells.

To determine the effect of 1% DMSO on the fungal cells, a control well containing 1% DMSO in RPMI 1640 with no drug was run in parallel to all test wells containing DMSO. In addition, all testing involved sterility control wells which were not inoculated, as well as growth control wells containing no antifungal agent.

Biofilm Growth on the device surface: Each Candida species formed biofilms on each peg of the device to achieve 10⁵ to 10⁶ CFU/peg after 22 hours after inoculation. C. glabrata was the most fastidious, requiring 10% CO₂ to establish biofilms. After 20 hours, C. glabrata grew only to about 5×10³ CFU/peg when incubated aerobically, while it grew to an average of 4.1×10⁴ CFU/peg in 10% CO₂. Aspergillus biofilms seemed to adhere more tightly to the pegs. Aspergillus grew to 10⁵ CFU/peg after 24 hour incubation (data not shown).

The biofilm that formed on the pegs of the MBEC device was similar for all species of Candida. Candida cells uniformly coated the entire peg and were encased is an extensive exopolysacchalide matrix. The Caclida cells grew as raised clusters of elongated cells in certain regions. Aspergillus biofilms were composed of organized conidiophores which swarmed over the entire peg after 24 hours. Exopolysaccharide was attached to the peg surface and surrounded each Aspergillus conidiophore.

Anti-fungal Susceptibility: The concentration of antibiotic required to inhibit planktonic cells (MIC), kill planktonic cells (MFC) and kill biofilm fungi (MBEC) are summarized in Table 3. The MIC and MFC values obtained from the NCCLS protocol and planktonic cells released from the biofilms which formed on the device pegs were similar or identical. The MIC and MFC obtained from the device were highly reproducible. Fungal biofilms were universally more difficult to eliminate than planktonic cells (Table 3).

Similar results were obtained for all species of Candida tested. In general the MBEC exceeded the MIC or MFC by several orders of magnitude. Although Amphoteracin B, nystatin, 5-fluorocytosine, fluconazole, itraconazole and ketoconazole were all effective in killing planktonic Candida cells only nystatin was effective against the same Candida growing as a biofilm. For example, amphotericin B, a polyene antifungal drug, was found to have an average MIC of 0.09 μg/ml and an average MFC of 0.02 μg/ml when used against planktonic cultures of C. albicans. Conversely, an average of 12 μg/ml of the same drug was required to kill biofilm cells of C. albicans. Griseofulvin and Polymyxin B were ineffective against planktonic and sessile Candida.

The MIC of Aspergillus fuimigatus, could not be obtained due to the clumping of Aspergillus cells in the 96 well microtiter plate, which renders analysis by the plate reader inaccurate. The MFC values (gathered by spot plating 100 μl of the well contents onto Rose Bengal Agar) demonstrated sensitivity of planktonic Aspergillus to amphotericin B, itraconazole, ketoconazole, and nystatin (Table 1). In contrast, none of the antifungal agents were effective against A. fumigatus biofilms even at the highest concentrations tested.

Azole drugs inhibited, but did not kill biofilm cells even at extremely high concentrations. Survival of viable cells is not a favorable result following drug therapy, and may contribute to the rise in azole-resistant strains of Candida (8). One may speculate that the failure of these drugs to eliminate biofilm cells is that they must be actively taken up by the cell. The decreased rate of drug uptake or inhibition of the exopolysaccharide by these biofilm organisms may prevent the drug from reaching its target enzyme. A fluconazole MIC less than or equal to 8 μg/ml against Candida species indicates that the species is susceptible to the drug (16). The C. albicans and C. tropicalis strains tested would be classified as susceptible to fluconazole according to these criteria. However, the MBEC values for fluconazole and itraconazole (>1024 μg/ml) was well above the breakpoint of 64 μg/ml for both of these isolates, indicating that the biofilm cells are resistant. Although fluconazole and itraconazole are the only drugs for which these tentative breakpoints have been established, the same trend was seen for ketoconazole and 5-fluorocytosine. This may explain why azoles are frequently ineffective in treating some cases of chronic candidiasis and Candida associated with implanted devices (4,5,12).

The polyenes, nystatin and amphotericin B, were the most effective agents in elimination of Candida biofilms. However, the MBEC of 16 μg/ml for amphotericin B may not be achievable under clinical situations—peak pennissible human serum concentrations are 2 μg/ml. The ability of the polyenes to work on the plasma membrane of fungi, without requiring uptake into the cell, may explain their relative effectiveness among the drugs tested against biofilm cells.

Once a protein surface such as L-lysine was provided, the Aspergillus readily formed an organized biofilm on the surface of the peg. The morphological features of the Aspergillus biofilm are not unlike that which occurs within tissue and on medical devices. Although the biofilm rapidly formed, it was still resistant to all agents tested. As with the Candida biofilms, it appears that growth rate does not influence resistance. An extensive exopolysaccharide was observed in the Aspergillus biofilms, which may be important in resistance. The crude mortality rate of patients treated with amphoteracin B for invasive pulmonary, sinus and cerebral aspergillosis has been reported to be 86%, 66% and 99% respectively. Only 54% of cases show any response to 14 days of treatment. Although polyenes show in vitro efficacy to Aspergillus they are largely infective for the treatment of invasive aspergillosis. The results of the MBEC assay would predict this treatment failure. Although to date there are no alternative chemotherapeutic agents for elimination of A. fumigatus, the assembly may be used for screening agents which could be potentially developed for future therapies.

It is established that success of a drug in vitro cannot be extrapolated to success in treatment therapy, but that failure of a drug in vitro should predict therapeutic failure. From this study, the azoles, 5-fluorocytosine, griseofulvin, and polymyxin B would be predicated to be unsuccessful in treating biofilm Candida infections, while the polyenes may or may not be effective.

What is clear from the research is that Candida and Aspeigillus species adhere to plastics, and that the formation of a biofilm tends to allow these organisms to withstand exposure to antimicrobial agents in concentrations many times greater than the same species grown in batch culture. It is no longer satisfactory to characterize antifungal agents against organisms in batch cultures when they are capable of growth in biofilms. In order to accurately assess the ability of a particular-agent to clear an infection of a biofilm organism, the susceptibility testing should be carried out on the cells as they would be found to exist in the host or in nature, i.e., displaying a profoundly altered physiology and encased in a protective exopolysaccharide matrix.

### Example 11

As noted above, a device of the present invention may be loaded with one or more anti-biofilm agents. An incomplete and exemplary list of possible anti-biofilm agents include, but are not limited to: Antibiotics. Including, but not limited to the following classes and members within a class: Aminoglycosides, such as Gentamicin, Tobramycin, Netilmicin, Amikacin, Kanamycin, Streptomycin, Neomycin, Quinolones/Fluoroquinolones, Nalidixic Acid, Cinoxacin, Norfloxacin, Ciprofloxacin, Perfloxacin, Ofloxacin, Enoxacin, Fleroxacin, and Levofloxacin; Antipseudomonal, such as Carbenicillin, Carbenicillin Indanyl, Ticarcillin, Azlocillin, Mezlocillin, Piperacillin Cephalosporins, Cephalothin, Cephaprin, Cephalexin, Cephradine, Cefadroxil, Cefazolin Cefamandole, Cefoxitin, Cefaclor, Cefuroxime, Cefotetan, Ceforanide, Cefuroxine Axetil, Cefonicid Cefotaxime, Moxalactam, Ceftizoxime, Ceftriaxone, Cefoperazone, Cftazidime, Other Cephalosporins, such as Cephaloridine, and Cefsulodin; other beta.-Lactam; Antibiotics, such as Imipenem, Aztreonam beta.-Lactamase Inhibitors Clavulanic Acid, Augmentin, Sulbactam; Sulfonamides, such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole; Urinary Tract Antiseptics, such as Methenamine, Nitrofurantoin, Phenazopyridine and other napthpyridines; Penicillins, such as Penicillin G and Penicillin V, Penicillinase Resistant Methicillin, Nafcillin, Oxacillin, Cloxacillin, Dicloxacillin Penicillins for Gram-Negative/Amino Penicillins Ampicillin (Polymycin), Amoxicillin, Cyclacillin, Bacampicillin; Tetracyclines, such as Tetracycline, Chlortetracycline, Demeclocycline, Methacycline, Doxycycline, Minocycline; other Antibiotics, such as Chlidramphenicol (Chlommycetin), Erythromycin, Lincomycin, Clindamycin, Spectinomycin, Polymyxin B (Colistin), Vancomycin, Bacitracin; Tuberculosis Drugs, such as Isoniazid, Rifampin, Ethambutol, Pyrazinamide, Ethinoamide, Aminosalicylic Acid, Cycloserine; Anti-Fungal Agents, such as Amphotericin B, Cyclosporine, Flucytosine Imidazoles and Triazoles Ketoconazole, Miconazaole, Itraconazole, Fluconazole, Griseofulvin; Topical Anti Fungal Agents, such as Clotrimazole, Econazole, Miconazole, Terconazole, Butoconazole, Oxiconazole, Sulconazole, Ciclopirox Olamine, Haloprogin, Tolnaftate, Naftifine, Polyene, Amphotericin B, Natamycin

### REFERENCES

1. Costerton J W, Stewart P S, Greenberg EP. 1999 Bacterial biofilms: a common cause of persistent infections. Science 284: 1318-1322
2. Costerton, J. W., K. Cheng, G. Geesey, T. Ladd, J. Nickel, M. Dasgupta and T. Marrie. 1987. Bacterial biofilms in nature and disease. Ann. Rev. Microbiol. 41: 435-64.
3. Ceri H., Olson M. E., Stremick C., Read R. R., Morck D. W., Buret A. G. 1999 The Calgary biofilm device: a new technology for rapid determination of antibiotic susceptabilities of bacterial biofilms. J Clin Microbiol 37: 1771-1776.
4. Maki D. G. Infections caused by Intravascular Devices used for infusion therapy: pathogenesis, prevention, and management. P155-212. In A. Bisno and F. Waldvogel (ed.) Infections Associated with Indwelling Medical Devices, 2^{nd} ed.-1994, American Society for Microbiology, Washington, D.C.
5. Tunney M. M., S. P Gorman. and S. Patrick. 1996. Infection associated with medical devices. Rev. Med. Microbiol. 7: 195-205
6. Fridkin, S K and W. R. jarvis. 1996. Epidemiology of nosocomial fungal infections. Clin Microbiol Rev 9: 499-511.
7. Latage J.-P. (1999) Aspirgillus fumigatus and aspirgillois. Clinical Microbiol Reviews 12: 310-350.
8. Ghannoum M A, Rice L. B. 1999 Antifungal agents: mode of action, mechanism of resistance and correlation of these mechanisms with bacterial resistance. Clin Microbiol Rev 12: 501-517.
9. National Committee for Clinical Laboratory Standards. 1995. Reference method for broth dilution antifungal susceptibility testing of yeasts. Tentative standard M27-T. National Committee for Clinical Laboratory Standards, Villanova, Pa.
10. Hawser, S. P., and L. Douglas. 1993. Biofilm formations by Candida species on the surface of catheter materials in vitro. Infection and Immunity. March: 915-21.
11. Hawser, S. P. and L. Douglas. 1995. Persistance of Candida albicans biofilms to antifungal agents in vitro. Antimicrob. Agents. Chemother. 39: 9, 2128-31.
12. Hawser S. P., G. S. Baillie and L. J. Douglas. 1998. Production of exopolysaccharide matrix by Candida albicans biofilms. J. Med. Microbiol. 47: 253-256.
13. Baillie G. S., Douglas L. J. 1998 Effect of growth rate on resistance of Candida albicans biofilms to antifungal agents. Antimicrob Agents Chemother 42: 1900-1905.
14. Baillie G. S., Douglas L. J. 1998 Iron-limited biofilms of Candida albicans and their susceptibility to Amphotericin B. Antimicrob Agents Chemother 42: 1900-1905.
15. Odds, F. C., 1988. Candida and Candidiosis. A review and bibliography. 2^{nd} Ed. Balliere Tindall, London, England.
16. Rex, J. H., M. Pfaller, J. Galggiani, M. Bartlett, A. Espinel-Ingroff, M. Lancaster, F. Odds, M. Rinaldi, T. Walsh and A. Bany. 1997. Development of interpretive breakpoints for antifungal susceptibility testing: Conceptual framework and analysis of in vitro—in vivo correlation data for fluconazole, itraconazole, and Candida infections. Clin. Infec. Dis. 24: 235-47.
17. Bodey, G. P. 1993. Candidiasis: Pathogenesis, Diagnosis, and Treatment. Raven Press Ltd. New York, N.Y.

Although a few preferred embodiments have been described, it will be appreciated by those skilled in the art that various changes and modifications might be made without departing from the scope of the invention. The terms and expressions in the preceding specification have been used therein as terms of description and not of limitation, and there is no intention in the use of such terms and expressions of excluding equivalents of the features shown and described or portions thereof, it being recognized as the scope of the invention as defined and limited only by the claims that follow.

We claim:

1. A method for determining the MIC, MBEC, and MBC values for a biofilm comprising: a) growing a biofilm, wherein said growing includes subjecting the biofilm to shear forces; b) exposing the grown biofilm to one or more anti-biofilm agents on a challenge plate; c) removing and neutralizing the exposed biofilm on a recovery plate; d) determining MIC values by evaluating any biofilm growth on the challenge plate; e) determining MBEC values by evaluating any biofilm growth on the recovery plate; and f) determining MBC values by evaluating viable cell counts from the recovery plate.

2. A method for treating one or more biofilm s comprising selecting an effective combination of anti-biofilm agents, wherein an effective combination consists essentially of two or more active agents of the same or different dose; contacting the biofilm with the combination of anti-biofilm agents; and determining the MIC, MBEC, MBC, or combinations thereof, for each combination.

3. The method of claim 1 wherein MIC, MBEC, and MBC values are determined.

4. The method of claim 1 further comprising, after the contacting step, neutralizing the anti-biofilm agent.

5. The method of claim 3, further comprising disrupting and collecting the biofilm.

6. An MBEC assay for testing sessile microorganisms comprising forming a biofilm on a pre-selected first plate; removing the biofilm and placing said biofilm on a second plate; exposing the biofilm to at least one active agent; and determining the effectiveness of said active agent against said biofilm.

7. An assay protocol for determining the MIC, MBEC, and MBC values for a biofilm comprising: 1) providing a first assembly having a first base and a first lid having projections extending therefrom, said first assembly being configured for growing one or more biofilms on one or more projections 2) providing a second assembly comprising a second base and the first lid, said second assembly being configured for exposing the biofilm on the projections to one or more anti-biofilm agents; 3) providing a third assembly comprising a third base and the first lid, said third assembly being configured for rinsing any biofilm on the projections; and 4) a fourth assembly comprising a fourth base and the first lid, said fourth assembly being configured and adapted to remove the biofilm from the projections.

8. The assay protocol of claim 7 comprising providing a first assembly having a first base pre-loaded with a nutrient composition.

9. The assay protocol of claim 7 comprising providing a second assembly having a second base pre-loaded with one or more biofilm agents.

10. The assay protocol of claim 7 comprising providing a third assembly having a third base pre-loaded with a rinsing composition.

11. The assay protocol of claim 7 comprising providing a fourth assembly having a fourth base pre-loaded with a recovery composition.
